(19) United States
(12) Patent Application Publication
(10) Pub. No.: US 2018/0310890 A1
(43) Pub. Date: Nov. 1, 2018

(54) METHODS AND SYSTEMS FOR DISEASE MONITORING AND ASSESSMENT

(71) Applicant: Coyote Bioscience Co, Ltd., Beijing (CN)

(72) Inventor: Xiang Li, Beijing (CN)

(21) Appl. No.: 15/947,641

(22) Filed: Apr. 6, 2018

Related U.S. Application Data
(63) Continuation of application No. PCT/CN2016/105441, filed on Nov. 11, 2016.
Application No. PCT/CN2016/105441 is a continuation-in-part of application No. PCT/CN2015/094425, filed on Nov. 12, 2015.

(51) Int. Cl. A61B 5/00; G06F 17/30; G16H 50/80; A61B 5/11; A61B 5/117

(52) U.S. Cl. CPC A61B 5/7275; G06F 17/30241; G06F 17/30864; G16H 50/80; A61B 5/7475; A61B 5/117; A61B 5/7435; A61B 5/746; A61B 5/4842; A61B 5/1112

(57) ABSTRACT

The present disclosure provides methods and systems for analyzing biological samples and information associated with a disease.

### CROSS-REFERENCE

This application is a continuation of PCT Application Serial No. PCT/CN2016/105441, filed Nov. 11, 2016, which is a continuation-in-part of PCT Patent Application No. PCT/CN2015/094425, filed Nov. 12, 2015, which is entirely incorporated herein by reference.

### BACKGROUND

The health or wellbeing of a subject may be determined by the subject's physical attributes and the environment(s) the subject encounters. For example, if the subject is exposed to a high concentration of a given virus at the subject's workplace, the subject may contract an illness. As another example, the subject may be exposed to a virus when the person is in proximity to another individual that carries the virus, which may lead the subject to contract an illness.

Conventional methods and systems for diagnosing and/or treating a disease condition may suffer a number of drawbacks. For example, such systems and methods may not be capable of drawing a relationship between the subject's environment and the subject's disposition in space and time. If the subject is exposed to a high concentration of a pathogen, the subject may not be able to detect the exposure and seek measures to prevent the onset of any potential disease condition. In addition, approaches for diagnosing and treating the subject may not be capable of pinpointing the time point at which the subject was exposed to a given pathogen. Such information may be crucial in identifying the type of pathogen that the subject was exposed to and providing a targeted remedy.

### SUMMARY

Risk assessment and monitoring of disease may be critical components of disease management. However, both risk assessment and monitoring of disease can rely on relatively isolated data sets that do not consider a number of items such as identity, physiological state, a given geographical location or a number of geographical locations. Accordingly, there can be substantial inaccuracies in both risk assessment and disease monitoring that can result in misdiagnosis of disease, underestimation or overestimation of risk and ultimately greater spread of disease than would otherwise occur. This is especially true in the case of infectious diseases, such as influenza or other pathogenic diseases that can give rise to an epidemic. Thus, there exists a need for rapid, accurate methods and systems for risk assessment and disease monitoring. Understanding the prevalence of and pin-pointing the location(s) and/or source(s) of an epidemic in real-time can permit both individuals and medical professionals to take quicker preventative and/or treatment actions when present in the location.

Recognized herein is the need for rapid, accurate methods and systems for risk assessment and disease monitoring. Understanding the prevalence of and pin-pointing the location(s) and/or source(s) of an epidemic in real-time can permit both individuals and medical professionals to take quicker preventative and/or treatment actions when present in the location.

The present disclosure provides methods and system for risk assessment and monitoring of disease. In some cases, assessment and/or monitoring include analysis that considers a geographic location or a plurality of geographic locations. Such analysis can also consider one or more quantitative measures of a biological marker Moreover, methods and systems described herein can be useful in obtaining disease information regarding the regression and/or progression of a disease and/or trends associated with a disease in the geographic location and/or the plurality of geographic measures. Such information can be provided to a user on an electronic display of an electronic device and can be useful in taking preventive and/or treatment actions with respect to an analyzed disease.

An aspect of the disclosure provides a method for providing a user with an assessment of a risk of contracting at least one disease. The method includes receiving, over a network, a search query of a user, where the search query includes information related to at least any two of an identity, a geographic location and a physiological state of the user; and processing, with the aid of a computer processor, the search query to identify one or more tags that are usable for searching in a disease database. The disease database can include an indication of the at least one disease; disease progression information that is indicative of a progression or regression of the at least one disease in one or more geographic locations; subject information selected from two or more of an identity, geographic location and physiological state of each of a plurality of subjects; and one or more associations between the at least one disease, disease progression information and subject information. The method also includes searching the disease database using the one or more tags to identify the at least one disease and the disease progression information; and based on the disease progression information, providing the user with the assessment of the risk of contracting the at least one disease.

In some embodiments, the user is provided with the assessment of the risk of contracting the at least one disease on a graphical user interface on an electronic display of an electronic device. In some embodiments, electronic device is a portable electronic device. In some embodiments, the graphical user interface is provided by a mobile computer application. In some embodiments, the information is related to the identity, geographic location and physiological state of the user. In some embodiments, the assessment is provided via a notification or alert over the network. In some embodiments, providing the user with the assessment comprises providing the user with one or more suggested preventative measures that reduce a rate of progression of the at least one disease in the geographic location.

In some embodiments, the indication of the at least one disease comprises identifying information for at least one virus, at least one bacterium and/or at least one protozoan. In some embodiments, the at least one virus is human immunodeficiency virus I (HIV I), human immunodeficiency virus II (HIV II), orthomyxovirus, Ebola virus, Dengue virus, influenza virus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox, adenovirus, Varicella Zoster virus, Human papilloma virus (HPV), Human T-cell Leukemia Virus (HTLV), mumps virus, Respiratory Syncytial Virus (RSV), parainfluenza virus or Rubella virus. In some embodiments, the at least one bacterium is Bordetella pertussis, Chlamydia pneumoniae, Chlamydia trachomatis, Campylobacter jejuni, Helicobacter pylori, Borrelia bacteria, Mycoplasma pneumoniae, Mycobacterium tuberculosis Haemophilus influenzae, Streptococcus pyogenes, Streptococcus pneumoniae, Clostridium tetani, Treponema pallidum, Trypanosoma cruzi, Toxoplasma gondii or Yersinia pestis. In some embodiments, the at least one protozoan is Plasmodium or Leishmania donovani.

In some embodiments, the identity includes at least one of a name, age and sex of the user. In some embodiments, the physiological state includes at least one of a heart rate, blood pressure, coughing frequency, coughing intensity, sneezing frequency, sneezing intensity, a level of chest congestion, a level of nasal congestion, body temperature, sweat level, weight, height, breathing rate, blood pressure, nerve conduction velocity, lung capacity, urine production rate, defecation rate, the presence of enlarged lymph nodes and a biochemical profile of a bodily fluid of the user.

In some embodiments, the geographic location is a continent, an island, a grouping of islands, a city/town/village, a county/township, a prefecture, a parish, a province, a state, a territory, an administrative region, a country, and/or a grouping of countries. In some embodiments, the geographic location is a region within the continent, the island, the grouping of islands, the city/town/village, the county/township, the prefecture, the parish, the province, the state, the territory, the administrative region, the country, and/or the grouping of countries.

An additional aspect of the disclosure provides a method for monitoring at least one disease in a subject. The method includes processing biological samples obtained directly from the subject at multiple time points to identify one or more biological markers in the biological samples and obtain a quantitative measure of at least a subset of the one or more biological markers across the multiple time points. Each of the one or more biological markers is indicative of a presence of the at least one disease in the subject and the processing is performed using nucleic acid amplification on each of the biological samples at a sample volume that is less than or equal to about 1 milliliter (mL) and in a time period that is less than or equal to about 10 minutes. The method also includes, with the aid of a computer processor, processing the quantitative measure to determine disease information indicative of a progression or regression of the at least one disease in the subject; and generating an output of the disease information. In some embodiments, the at least one disease is monitored in a fixed geographic location.

In some embodiments, each of the biological samples is obtained directly from the subject and processed without subjecting the biological samples to purification to isolate the one or more biological markers. In some embodiments, the biological samples comprise whole blood. In some embodiments, the biological samples comprise saliva. In some embodiments, the biological samples comprise urine. In some embodiments, the biological samples comprise sweat. In some embodiments, the biological samples are processed without nucleic acid extraction from the biological samples.

In some embodiments, the nucleic acid amplification comprises polymerase chain reaction (PCR). In some embodiments, the nucleic acid amplification comprises reverse transcription polymerase chain reaction (RT-PCR). In some embodiments, the processing the biological samples comprises providing a reaction vessel comprising a given biological sample of the biological samples and reagents necessary for conducting nucleic acid amplification; and subjecting the given biological sample to nucleic acid amplification under conditions that are sufficient to yield an amplification product that is indicative of a presence of the one or more biological markers. In some embodiments, the reagents comprise a polymerizing enzyme. In some embodiments, the reagents comprise one or more primers having sequence complementary with the one or more biological markers. In some embodiments, the nucleic acid amplification comprises reverse transcription in parallel with deoxyribonucleic acid (DNA) amplification. The reagents can include a reverse transcriptase, a DNA polymerase, and a primer set for a ribonucleic acid (RNA) indicative of the at least one disease.

In some embodiments, processing the quantitative measure comprises comparing the quantitative measure at the multiple time points to a reference to identify the progression or regression of the at least one disease in the subject. In some embodiments, the one or more biological markers comprise a nucleic acid. In some embodiments, the nucleic acid is derived from a virus. In some embodiments, the virus is human immunodeficiency virus I (HIV I), human immunodeficiency virus II (HIV II), orthomyxovirus, Ebola virus, Dengue virus, influenza virus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox, adenovirus, Varicella Zoster virus, Human papilloma virus (HPV), Human T-cell Leukemia Virus (HTLV), mumps virus, Respiratory Syncytial Virus (RSV), parainfluenza virus or Rubella virus. In some embodiments, the nucleic acid is derived from a bacterium. In some embodiments, the bacterium is Bordetella pertussis, Chlamydia pneumoniae, Chlamydia trachomatis, Campylobacter jejuni, Helicobacter pylori, Borrelia bacteria, Mycoplasma pneumoniae, Mycobacterium tuberculosis, Haemophilus influenzae, Streptococcus pneumoniae, Streptococcus pyogenes, Clostridium tetani, Treponema pallidum, Trypanosoma cruzi, Toxoplasma gondii or Yersinia pestis. In some embodiments, the nucleic acid is derived from a protozoan. In some embodiments, the protozoan is Plasmodium or Leishmania donovani.

In some embodiments, each of the biological samples is processed in a time period that is less than or equal to about 5 minutes. In some embodiments, each of the biological samples is processed in a time period that is less than or equal to about 2 minutes. In some embodiments, each of the biological samples is processed in a time period that is less than or equal to about 1 minute. In some embodiments, each of the biological samples is processed in a time period that is less than or equal to about 0.5 minutes.

In some embodiments, the sample volume is less than or equal to about 0.5 mL. In some embodiments, the sample volume is less than or equal to about 0.1 mL. In some embodiments, the sample volume is less than or equal to about 0.01 mL.

In some embodiments, generating the output comprises providing the disease information to a user on a graphical user interface of an electronic display. In some embodiments, the graphical user interface is provided by a mobile computer application. In some embodiments, the user is the subject. In some embodiments, the user is a healthcare professional. In some embodiments, generating the output comprises transmitting the disease information to a remote data storage unit.

In some embodiments, the method further comprises providing the subject with a questionnaire to assess a geographic location and/or physiological state of the subject; and identifying the at least one disease from results of the questionnaire. In some embodiments, the questionnaire is provided to the subject on a user interface of an electronic device. In some embodiments, the user interface is provided by a mobile computer application. In some embodiments, the method further comprises drawing a correlation(s) between results of the questionnaire and the at least one disease.

An additional aspect of the disclosure provides a method for monitoring at least one disease. The method includes receiving, over a network, disease information for each of a plurality of subjects. For a given subject of the plurality of subjects, the disease information is generated by: processing biological samples obtained directly from the given subject at multiple time points to identify one or more biological markers in the biological samples, where each of the one or more biological markers is indicative of a presence of the at least one disease in the given subject, and where the processing is performed using nucleic acid amplification on each of the biological samples at a sample volume that is less than or equal to about 1 milliliter (mL) and in a time period that is less than about 10 minutes; obtaining a quantitative measure of at least a subset of the one or more biological markers across the multiple time points; and with the aid of a computer processor, processing the quantitative measure to determine the disease information, where the disease information is indicative of a progression or regression of the at least one disease in the given subject. The method further comprises compiling the disease information in a memory location; processing the compiled disease information to identify a trend of the disease in a given geographic location and/or across a plurality of geographic locations; and generating an output indicative of the trend.

In some embodiments, each of the biological samples is obtained directly from the subject and processed without subjecting the biological samples to purification to isolate the one or more biological markers. In some embodiments, the biological samples comprise whole blood. In some embodiments, the biological samples comprise saliva. In some embodiments, the biological samples comprise urine. In some embodiments, the biological samples comprise sweat. In some embodiments, the biological samples are processed without nucleic acid extraction from the biological samples.

In some embodiments, the nucleic acid amplification comprises polymerase chain reaction (PCR). In some embodiments, the nucleic acid amplification comprises reverse transcription polymerase chain reaction (RT-PCR). In some embodiments, the processing the biological samples comprises providing a reaction vessel comprising a given biological sample of the biological samples and reagents necessary for conducting nucleic acid amplification; and subjecting the given biological sample to nucleic acid amplification under conditions that are sufficient to yield an amplification product that is indicative of a presence of the one or more biological markers. In some embodiments, the reagents comprise a polymerizing enzyme. In some embodiments, the reagents comprise one or more primers having sequence complementary with the one or more biological markers. In some embodiments, the nucleic acid amplification comprises reverse transcription in parallel with deoxyribonucleic acid (DNA) amplification. The reagents can include a reverse transcriptase, a DNA polymerase, and a primer set for a ribonucleic acid (RNA) indicative of the at least one disease.

In some embodiments, processing the quantitative measure comprises comparing the quantitative measure at the multiple time points to a reference to identify the progression or regression of the at least one disease in the subject. In some embodiments, the one or more biological markers comprise a nucleic acid. In some embodiments, the nucleic acid is derived from a virus. In some embodiments, the virus is human immunodeficiency virus I (HIV I), human immunodeficiency virus II (HIV II), orthomyxovirus, Ebola virus, Dengue virus, influenza virus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox, adenovirus, Varicella Zoster virus, Human papilloma virus (HPV), Human T-cell Leukemia Virus (HTLV), mumps virus, Respiratory Syncytial Virus (RSV), parainfluenza virus or Rubella virus. In some embodiments, the nucleic acid is derived from a bacterium. In some embodiments, the bacterium is Bordetella pertussis, Chlamydia pneumoniae, Chlamydia trachomatis, Campylobacter jejuni, Helicobacter pylori, Haemophilus influenza, Borrelia bacteria, Mycoplasma pneumoniae, Mycobacterium tuberculosis, Streptococcus pneumoniae, Streptococcus pyogenes, Clostridium tetani, Treponema pallidum, Trypanosoma cruzi, Toxoplasma gondii and Yersinia pestis. In some embodiments, the nucleic acid is derived from a protozoan. In some embodiments, the protozoan is Plasmodium and Leishmania donovani.

In some embodiments, each of the biological samples is processed in a time period that is less than or equal to about 5 minutes. In some embodiments, each of the biological samples is processed in a time period that is less than or equal to about 2 minutes. In some embodiments, each of the biological samples is processed in a time period that is less than or equal to about 1 minute. In some embodiments, each of the biological samples is processed in a time period that is less than or equal to about 0.5 minutes.

In some embodiments, the sample volume is less than or equal to about 0.5 mL. In some embodiments, the sample volume is less than or equal to about 0.1 mL. In some embodiments, the sample volume is less than or equal to about 0.01 mL.

In some embodiments, generating the output comprises providing the trend to a user on a graphical user interface of an electronic display. In some embodiments, the graphical user interface is provided by a mobile computer application. In some embodiments, the user is a given subject of the plurality of subjects. In some embodiments, the user is a healthcare professional. In some embodiments, generating the output comprises storing the trend in a memory location. In some embodiments, generating the output comprises providing a notification or alert to a user with respect to the trend. In some embodiments, the biological samples are processed at a designated point-of-care device among a plurality of point-of-care devices.

In some embodiments, generating the output comprises providing an update with respect to the trend. In some embodiments, the update is indicative of an increase in a prevalence of the at least one disease. In some embodiments, the update is indicative of a decrease in a prevalence of the at least one disease. In some embodiments, the trend of the disease is in a given geographic location. In some embodiments, each of the plurality of subjects is located at the given geographic location. In some embodiments, the trend of the disease is across a plurality of geographic locations. In some embodiments, each of the plurality of subjects is located at a given geographic location of the plurality of geographic locations.

An additional aspect of the disclosure provides a non-transitory computer-readable medium comprising machine-executable code that, upon execution by one or more computer processors, implements method for providing a user with an assessment of a risk of contracting at least one disease. The method includes receiving, over a network, a search query of a user that includes information related to at least any two of an identity, a geographic location and a physiological state of the user; processing, with the aid of a computer processor, the search query to identify one or more tags that are usable for searching in a disease database. The disease database comprises an indication of the at least one disease; disease progression information that is indicative of a progression or regression of the at least one disease in one or more geographic locations; subject information selected from two or more of an identity, geographic location and physiological state of each of a plurality of subjects; and one or more associations between the at least one disease, disease progression information and subject information. The method further comprises searching the disease database using the one or more tags to identify the at least one disease and the disease progression information; and based on the disease progression information, providing the user with the assessment of the risk of contracting the at least one disease.

An additional aspect of the disclosure provides a non-transitory computer-readable medium comprising machine-executable code that, upon execution by one or more computer processors, implements method for providing a user with an assessment of a risk of contracting at least one disease. The method includes processing biological samples obtained directly from the subject at multiple time points to identify one or more biological markers in the biological samples; and obtain a quantitative measure of at least a subset of the one or more biological markers across the multiple time points. Each of the one or more biological markers is indicative of a presence of the at least one disease in the subject and the processing is performed using nucleic acid amplification on each of the biological samples at a sample volume that is less than or equal to about 1 milliliter (mL) and in a time period that is less than or equal to about 10 minutes. The method further comprises, with the aid of a computer processor, processing the quantitative measure to determine disease information indicative of a progression or regression of the at least one disease in the subject; and generating an output of the disease information.

An additional aspect of the disclosure provides a non-transitory computer-readable medium comprising machine-executable code that, upon execution by one or more computer processors, implements method for providing a user with an assessment of a risk of contracting at least one disease. The method includes receiving, over a network, disease information for each of a plurality of subjects. For a given subject of the plurality of subjects, the disease information is generated by: processing biological samples obtained directly from the given subject at multiple time points to identify one or more biological markers in the biological samples, where each of the one or more biological markers is indicative of a presence of the at least one disease in the given subject, and where the processing is performed using nucleic acid amplification on each of the biological samples at a sample volume that is less than or equal to about 1 milliliter (mL) and in a time period that is less than about 10 minutes; obtaining a quantitative measure of at least a subset of the one or more biological markers across the multiple time points; and with the aid of a computer processor, processing the quantitative measure to determine the disease information, where the disease information is indicative of a progression or regression of the at least one disease in the given subject. The method further comprises compiling the disease information in a memory location; processing the compiled disease information to identify a trend of the disease in a given geographic location and/or across a plurality of geographic locations; and generating an output indicative of the trend.

Another aspect of the present disclosure provides a non-transitory computer-readable medium comprising machine-executable code that, upon execution by one or more computer processors, implements any of the methods above or elsewhere herein.

Another aspect of the present disclosure provides a computer system comprising one or more computer processors and a computer-readable medium coupled thereto. The computer-readable medium comprises machine-executable code that, upon execution by the one or more computer processors, implements any of the methods above or elsewhere herein.

In some aspects, the present disclosure involves providing a user with an assessment of a risk of contracting at least one disease while travelling. The present disclosure further involves optimizing an itinerary.

In one aspect among the some aspects, the present disclosure involves a method for providing a user with an assessment of a risk of contracting at least one disease, comprising: (a) receiving, over a network, a search query of a user, which search query includes information related to a destination, and optionally one or more waypoints; (b) processing, with the aid of a computer processor, the search query to identify one or more geographic location tags associated with the destination and optionally the one or more waypoints for searching in a disease database, wherein the disease database comprises disease progression information that is indicative of a progression or regression of the at least one disease in one or more geographic locations, including the destination; (c) searching the disease database using the one or more geographic location tags to identify the at least one disease and the disease progression information; and (d) based on the disease progression information identified in (c), providing the user with the assessment of the risk of contracting the at least one disease at the destination and, in some cases, the one or more waypoints.

In some embodiments, the user may be provided with the assessment of the risk of contracting the at least one disease on a graphical user interface on an electronic display of an electronic device.

In some embodiment, the electronic device may be a portable electronic device.

In some embodiment, the graphical user interface may be provided by a mobile computer application.

In some embodiment, the search query may further include an identity and/or physiological state of the user.

In some embodiment, the search query may include a starting point of the user.

In some embodiments, the assessment may be provided via a notification or alert over the network.

In some embodiments, providing the user with the assessment may comprise providing the user with one or more suggested preventative measures that reduce a rate of progression of the at least one disease in the destination and/or waypoints.

In some embodiments, providing the user with the assessment may comprise suggesting that the user avoid travelling to the destination.

In some embodiments, providing the user with the assessment may comprise suggesting that the user avoid travelling via at least one waypoint of the one or more waypoints.

In some embodiments, providing the user with the assessment may comprise suggesting that the user travel to a different destination.

In some embodiments, the database may further comprise an indication of the at least one disease.

In some embodiments, the indication of the at least one disease comprises identifying information for at least one virus, at least one bacterium and/or at least one protozoan.

In some embodiments, the at least one virus may be selected from the group consisting of human immunodeficiency virus I (HIV I), human immunodeficiency virus II (HIV II), orthomyxovirus, Ebola virus, Dengue virus, influenza virus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox, adenovirus, Varicella Zoster virus, Human papilloma virus (HPV), Human T-cell Leukemia Virus (HTLV), mumps virus, Respiratory Syncytial Virus (RSV), parainfluenza virus, Rubella virus, Zika virus, Middle East respiratory syndrome (MERS) Virus, Yellow Fever virus, Rift Valley fever virus, Chikungunya Fever virus, enterovirus, Cosksackie virus, and norovirus.

In some embodiments, the at least one bacterium may be selected from the group consisting of Bordetella pertussis, Chlamydia pneumoniae, Chlamydia trachomatis, Campylobacter jejuni, Helicobacter pylori, Borrelia bacteria, Mycoplasma pneumoniae, Mycobacterium tuberculosis Haemophilus influenzae, Streptococcus pyogenes, Streptococcus pneumoniae, Clostridium tetani, Treponema pallidum, Trypanosoma cruzi, Toxoplasma gondii, Yersinia pestis, and Salmonella sp.

In some embodiments, the at least one protozoan may be selected from the group consisting of Plasmodium and Leishmania donovani.

In some embodiments, the identity may include at least one of a name, age and sex of the user.

In some embodiments, the physiological state may include at least one of a heart rate, blood pressure, coughing frequency, coughing intensity, sneezing frequency, sneezing intensity, a level of chest congestion, a level of nasal congestion, body temperature, sweat level, weight, height, breathing rate, blood pressure, nerve conduction velocity, lung capacity, urine production rate, defecation rate, the presence of enlarged lymph nodes and a biochemical profile of a bodily fluid of the user.

In some embodiments, the method may further comprise providing the total risk of contracting the at least one disease of travelling via the waypoints to the destination.

In some embodiments, the search query may further include information regarding the itinerary of travelling via the waypoints to the destination.

In some embodiments, the itinerary may include the time of arrival at each waypoint or the destination, the time of departure from each waypoint or the starting point, and/or the time of stay at each waypoint.

In some embodiments, providing the user with the assessment of the risk of contracting the at least one disease in (d) may further comprise taking into account the itinerary.

In another aspect among the aspects, the present disclosure involves a method for providing a user with an assessment of a risk of contracting at least one disease, comprising: (a) receiving, over a network, a search query of a user, which search query includes information related to a starting point and a destination selected by the user; (b) processing, with the aid of a computer processor and a travel cost data structure, the search query to (i) identify a route leading from the starting point to the destination within the travel cost data structure, and (ii) determine one or more waypoints along the route, wherein the one or more waypoints include at least the starting point and the destination, and wherein the travel cost data structure comprises geographic locations and travel cost between neighboring geographic locations; (c) using the one or more waypoints to search a disease database comprising disease progression information that is indicative of a progression or regression of the at least one disease in one or more geographic locations, including the destination and/or the one or more waypoints, to identify the at least one disease and the disease progression information; and (d) based on the disease progression information identified in (c), providing the user with the assessment of the risk of contracting the at least one disease at the destination and/or along the route.

In some embodiments, the travel cost may include one or more members that are selected from the group consisting of travel time, travel expense, travel comfort level, residence time, predictability, safety, punctuality, and combinations thereof.

In some embodiments, the travel cost may include two or more members selected from the group, which two or more members are in a weighted combination.

In some embodiments, the travel cost data structure may be a weighted map comprising the geographic locations as vertices and the travel cost between neighboring geographic locations as weighted edges.

In some embodiments, the travel cost data structure may be a table comprising geographic locations in columns and rows and the travel cost between neighboring geographic locations in cells.

In some embodiments, the method may further comprise creating an itinerary based on the route.

In some embodiments, in (b), the route leading from the starting point to the destination within the travel cost data structure may be generated by employing a pathfinding algorithm over the travel cost data structure.

In some embodiments, the pathfinding algorithm may be selected from the group consisting of A*, Dijkstra, BFS, DFS, Greedy, and combinations thereof.

In another aspect among some aspects, the present disclosure involves a method for providing a user with an itinerary to a destination, comprising: (a) receiving, over a network, a search query of a user, which search query includes information related to a starting point and a destination selected by the user; (b) processing, with the aid of a computer processor and a travel cost data structure, the search query to (i) identify a route leading from the starting point to the destination within the travel cost data structure, and (ii) determine a plurality of waypoints along the route, wherein the plurality of waypoints includes at least the starting point and the destination, and wherein the travel cost data structure comprises geographic locations and travel cost between neighboring geographic locations; (c) using each waypoint of the plurality of waypoints to search a disease database comprising disease progression information that is indicative of a progression or regression of the at least one disease in one or more geographic locations to identify the at least one disease and the disease progression information associated with the waypoint of the plurality of waypoints; (d) based on the disease progression information identified in (c), (i) determining a risk of contracting the at least one disease, and (ii) optimizing the travel cost data structure by adjusting the travel cost between the geographic location associated with the waypoint and neighboring geographic locations based on the risk; (e) repeating (b) through (d), as necessary, to generate an optimum route, wherein the optimum route reduces the risk of contracting the at least one disease; and (f) using the optimum route in (e) to generate an itinerary for the user.

In some embodiments, the user may be provided with the itinerary on a graphical user interface on an electronic display of an electronic device.

In some embodiments, providing the user with the itinerary may further comprise providing the user with an assessment of a risk of contracting at least one disease.

In another aspect among some aspects, the present disclosure involves a method for providing a user with an itinerary to a destination, comprising: (a) receiving, over a network, a search query of a user, which search query includes information related to a starting point and a destination selected by the user; (b) processing, with the aid of a computer processor and a travel cost data structure, the search query to (i) identify a plurality of routes leading from the starting point to the destination within the travel cost data structure, and (ii) for each route of the plurality of routes, determine a plurality of waypoints along the route, wherein the plurality of waypoints includes at least the starting point and the destination, and wherein the travel cost data structure comprises geographic locations and travel cost between neighboring geographic locations; (c) for each route of the plurality of routes, using each waypoint of the plurality of waypoints to search a disease database comprising disease progression information that is indicative of a progression or regression of the at least one disease in one or more geographic locations to identify the at least one disease and the disease progression information associated with the waypoint of the plurality of waypoints; (d) based on the disease progression information identified in (c), for each route of the plurality of routes, (i) determining a risk of contracting the at least one disease along the route, and (ii) optimizing the travel cost data structure by adjusting the travel cost between the geographic location associated with the waypoint and neighboring geographic locations based on the risk; (e) repeating (b) through (d), as necessary, to generate an optimum route, wherein the optimum route incurs the lowest travel cost among the plurality of routes; and (f) using the optimum route in (e) to generate an itinerary for the user.

In another aspect among some aspects, the present disclosure involves a method for optimizing a travel cost data structure comprising a plurality of geographic locations and travel cost data structure between neighboring geographic locations, comprising: (a) using each geographic location of the plurality of geographic locations to search a disease database comprising disease progression information that is indicative of a progression or regression of at least one disease in one or more geographic locations, to identify at least one disease and disease progression information associated with the geographic location of the at least plurality of geographic locations; (b) based on the at least one disease and disease progression information identified in (a), (i) determining a risk of contracting the at least one, and (ii) optimizing the travel cost data structure by adjusting the travel cost between the each geographic location of the plurality of geographic locations and all geographic locations based on the risk; and (c) repeating (a) through (b) until all geographic locations of the plurality of geographic locations have been traversed, thereby optimizing the travel cost data structure.

In another aspect among some aspects, the present disclosure involves a method for providing a user with an itinerary to a destination using an optimized travel cost data structure, comprising: i. receiving, over a network, a search query of a user, which search query includes information related to a starting point and a destination selected by the user; ii. processing, with the aid of a computer processor and the optimized travel cost data structure, the search query to identify an optimum route leading from the starting point to the destination within the travel cost data structure; and iii. using the optimum route in ii. to generate an itinerary for the user.

In some embodiments, the method further comprises (a) using each waypoint of the one or more waypoints to search a disease database comprising disease progression information that is indicative of a progression or regression of the at least one disease in one or more geographic locations, including the destination, to identify the at least one disease and the disease progression information; and (b) based on the disease progression information identified in (a), providing the user with the assessment of the risk of contracting the at least one disease at the destination or along the route.

In some embodiments, providing the user with the assessment of the risk of contracting the at least one disease in (b) may further comprise taking into account the itinerary.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure”, “FIG.” and “FIG.” herein), of which:

FIG. 1 is a workflow of an example method for assessing the risk associated with contracting a disease;

FIG. 2 is a workflow of an example method for monitoring a disease in a subject;

FIG. 3 is a workflow of an example method for monitoring a disease;

FIG. 4 is a schematic representation of an example computer control system that can aid in implementing methods described herein; and

FIGS. 5A-5G are schematic depictions of various views of an example computer application that can be used in accordance with a method described herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the singular form “a”, “an” and “the” include plural references unless the context clearly dictates otherwise. For example, the term “a cell” includes a plurality of cells, including mixtures thereof.

As used herein, the term “about” generally refers to a range that is 15% greater than or less than a stated numerical value within the context of the particular usage. For example, “about 10” would include a range from 8.5 to 11.5.

As used herein, the terms “amplifying”, “amplification” and “nucleic acid amplification” are used interchangeably and generally refer to generating one or more copies or “amplified product” of a nucleic acid. The term “reverse transcription amplification” generally refers to the generation of deoxyribonucleic acid (DNA) from a ribonucleic acid (RNA) template via the action of a reverse transcriptase.

As used herein, the term “geographic location” generally refers to a particular position on the Earth or other celestial object. A geographic location can be described in any appropriate way including with geographic coordinates (e.g., latitude and longitude); with the name of a geographical region (e.g., a continent, an island, a grouping of islands, a region of a particular country, a region of a particular continent, a region of a particular country, state/province, city/town/village, etc., a region associated with a geographical feature such as a body of water, mountain range, dessert, plain, rainforest, etc.); with the name of a place such as a city/town/village, county/township, prefecture, parish, province, state, territory, administrative region, country, and/or grouping of countries (e.g., European Union, the United Kingdom); one or more demographic characteristics (e.g., as having a certain population, ethnic group, etc.) and with the name of a particular landmark such as a building, a school, a workplace, a shopping center, a community center, a religious institution, a hospital, a health clinic, a mobile unit, a humanitarian aid camp, a home, or a grouping of homes (e.g., a subdivision, an apartment community, a dormitory, etc.). In some cases, a geographic location can also be described by one or more of its characteristics (e.g., climate (e.g., precipitation, air temperature, air quality, UV-index, allergen levels, etc.). In some cases, a geographic location can be identified by its PM2.5 value, a measure of the amount of fine particles of up to 2.5 micrometers in size (e.g., diameter) in the geographic location's air.

Furthermore, in some cases, the geographic location can be determined automatically by an electronic device via, for example, the capability for accessing a global navigation satellite system, such as the global positioning system (GPS) system, the Globalnaya navigatsionnaya sputnikovaya Sistema (GLONASS), Indian Regional Navigation Satellite System (IRNSS), BeiDou Navigation Satellite System (BDS), Galileo (the European satellite navigation system), and the like.

Alternatively, the geographic location can be determined automatically by an electronic device via any one of a plurality of geolocation techniques other than the global navigation satellite system, such as multilateration of radio signals, Global System for Mobile Communication (GSM), location based services of a mobile device, Wi-Fi based location, hybrid positioning system, and the like.

As used herein, the term “identity” generally refers to a classification that describes a subject or a particular group to which a subject belong (e.g., a gender, an age group, an ethnic group, a disease group, etc.). Non-limiting examples of such classifications include a subject's name (e.g., one or more of a first name, a last name, a nickname, etc.), a subject's age (e.g., including within a particular age range) and the gender/sex (e.g., male, female, intersex, etc.). In some cases, identity is provided by a biometric measure such as a finger print, a retina scan, voice recognition and a nucleic acid sequence or combination of nucleic acid sequences unique to a particular individual.

As used herein, the term “nucleic acid” generally refers to a polymeric form of nucleotides of any length, either deoxyribonucleotides (dNTPs) or ribonucleotides (rNTPs), or analogs thereof. Nucleic acids may have any three dimensional structure, and may perform any function, known or unknown. Non-limiting examples of nucleic acids include DNA, RNA, coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent.

As used herein, the term “physiological state” generally refers to a collection of one or more measures indicative of with the physical condition of a subject. A physiological state can be made up of any collection of such measures, with non-limiting examples of such measures that include height, weight, heart rate, sneezing frequency, sneezing intensity, coughing frequency, coughing intensity, level of nasal congestion, level of chest congestion, blood pressure, body temperature, level of sweat production, nerve conduction velocity, breathing rate, lung capacity, urine production rate, defecation rate, the presence of enlarged lymph nodes, biochemical profile of a bodily fluid (e.g., blood biochemical profile, urine biochemical profile, saliva biochemical profile, etc.) and skin moisture content.

As used herein, the term “reaction mixture” generally refers to a composition comprising reagents necessary to complete nucleic acid amplification (e.g., DNA amplification, RNA amplification), with non-limiting examples of such reagents that include primer sets having specificity for target RNA or target DNA, DNA produced from reverse transcription of RNA, a DNA polymerase, a reverse transcriptase (e.g., for reverse transcription of RNA), suitable buffers (including zwitterionic buffers), co-factors (e.g., divalent and monovalent cations), dNTPs, and other enzymes (e.g., uracil-DNA glycosylase (UNG)), etc). In some cases, reaction mixtures can also comprise one or more reporter agents.

As used herein, the term “tag” generally refers to a word or string of words of a search query that, with the aid of a computer processor, can be recognized and used to search a database. In some cases, an equivalent word or string of words to a tag is stored in a database to be searched, with the tag recognized by the computer processor during searching as being a member of the database. A “geographic location tag” is a “tag” associated with a geographic location as described elsewhere herein.

As used herein, the term “target nucleic acid” generally refers to a nucleic acid molecule in a starting population of nucleic acid molecules having a nucleotide sequence whose presence, amount, and/or sequence, or changes in one or more of these, are desired to be determined. A target nucleic acid may be any type of nucleic acid, including DNA, RNA, and analogues thereof. As used herein, a “target ribonucleic acid (RNA)” generally refers to a target nucleic acid that is RNA. As used herein, a “target deoxyribonucleic acid (DNA)” generally refers to a target nucleic acid that is DNA. In some cases, a target nucleic acid may be indicative of one or more diseases.

As used herein, the term “subject,” generally refers to an entity or a medium that has testable or detectable information. A subject can be a person or individual. A subject can be a vertebrate, such as, for example, a mammal (e.g., human, dog, or cat) or a bird. Non-limiting examples of mammals include murines, simians, humans, farm animals (e.g., cows, chickens, horses, pigs, sheep, etc.), sport animals, and pets (e.g., dogs, cats, hamsters, rats, mice, guinea pigs, ferrets, etc.)

The present disclosure provides point-of-care (POC) systems for testing and analysis, which may improve the detection and management of infectious diseases in various settings, such as dense settings, resource-limited settings with poor laboratory infrastructure, or in remote areas where there are delays in the receipt of laboratory results and potential complications to following up with patients. POC methods and systems of the present disclosure may render health care facilities more capable of delivering sample-to-answer results to patients during a single visit. In addition, POC methods and systems of the present disclosure enable enhanced risk assessment and/or monitoring of diseases from a geographical standpoint, due to the availability of rapid communication networks, including wireless and satellite networks. POC devices capable of rapid communication via one of these networks can transmit data to remote computers (e.g., computer servers) that can compile data that can be searched by a user and/or used for disease risk assessment, disease monitoring and disease management.

In an aspect, the disclosure provides a method for providing a user with an assessment of a risk of contracting at least one disease. The method includes receiving, over a network, a search query of a user, which search query includes information related to at least any two of an identity, a geographic location and a physiological state of the user. With the aid of a computer, the search query is then processed to identify one or more tags that are usable for searching in a disease database. The disease database can include an indication of the at least one disease; disease progression information that is indicative of a progression or regression of the at least one disease in one or more geographic locations; subject information selected from two or more of an identity, geographic location, health state and physiological state of each of a plurality of subjects; and/or one or more associations between the at least one disease, disease progression information and subject information. Moreover, the method also includes searching the disease database using the one or more tags to identify the at least one disease and the disease progression information, and based on the disease progression information, providing the user with the assessment of the risk of contracting the at least one disease. In some cases, the search query includes information related to all three of identity, geographic location and physiological state of the user. In general, the user is a human.

The search query of the user can be provided to an electronic device that transmits the search query over the network for processing by the computer processor. Non-limiting examples of an electronic device include a personal computer (laptop computer, desktop computer, a video game console), a portable electronic device (e.g., a mobile telephone (e.g., a smartphone or the like capable of running mobile applications (apps)), a tablet computer, a pager, a calculator, a portable video game console, a portable music player (e.g., iPod™ or the like)). Additionally, the computer processor can be a component of a remote computer system networked with the electronic device. The network can be the Internet, an internet and/or extranet, or an intranet and/or extranet that is in communication with the Internet. In some cases, the network is a cellular phone network that is in communication with the Internet. In some cases, the remote computer system is a part of a decentralized computing network (e.g., a network “cloud”) comprising the remote computer system and, in some cases, the electronic device.

The disease database can be stored in the computer memory of a computer system, including an example computer system described elsewhere herein. Moreover, the disease database can be updatable in that regular updates can be made to the database, including in real-time. As is discussed above, the disease database includes an indication of a least one disease. Non-limiting examples of such an indication include identifying information for a disease (e.g., disease name), identifying information for at least one pathogen (e.g., a bacterial pathogen (including bacteria described elsewhere herein), a viral pathogen (including viruses described elsewhere herein)) associated with a disease, identifying information for at least one symptom associated with the disease and a biochemical profile (e.g., biochemical profile of a bodily fluid, biochemical profile of a tissue sample) associated with the disease.

As is discussed above, the disease database also includes disease progression information that is indicative of a progression or regression of the at least one disease in one or more geographic locations. Such information can include an incidence rate of the at least one disease in the one or more geographic locations; a longitudinal incidence rate or the at least one disease in the one or more geographic locations; a mortality rate of the at least one disease in the one or more geographic locations; a longitudinal mortality rate of the at least one disease in the one or more geographic areas; and/or the prevalence of one or more symptoms associated with the at least one disease in the one or more geographic areas. In some cases, the disease database may comprise a plurality of types of disease progression information.

The disease database also includes subject information selected from two or more of an identity, geographic location and physiological state of each of a plurality of subjects. Such information can be provided to the database statically (e.g., through one or more datasets available at a fixed point in time) or may be made in real-time, whereby subject data is continuously added to the database from users in communication with the database. Real-time updates can be provided to the disease database from input data received from various users of the disease database. In some cases, the subject information can be the same type of information related to the at least two of an identity, a geographic location and/or a physiological state of the user making the search query.

As is discussed above, the disease database also includes one or more associations between the at least one disease, disease progression information and subject information. Such associations include correlations between various disease database components. For example, the subject information may comprise data that indicate that a plurality of subjects in a particular neighborhood have a relatively high heart rate. The disease progression information may indicate that the incidence of the particular disease in neighborhood subjects having a relatively high heart rate has increased with time. In this example, the disease database could, thus, also include an association between the subjects having relatively high heart rate in the neighborhood and the increasingly high incidence rate of the disease amongst these individuals in the neighborhood. Any suitable combination of disease, disease progression information and subject information can be used to generate an association. In some cases, the disease database comprises a plurality of associations between the disease, disease progression information and subject information of the disease database.

Moreover, the disease database can be searched using the one or more tags to identify the at least one disease and the disease progression information. During processing, the computer processor can recognize tags in the search query of the user and find these tags stored in the disease database. The tags can be a component of an indication of the at least one disease and/or a component of disease progression information.

Based on the disease progression information identified from the disease database, the user can be provided with the assessment of the risk of contracting the disease. The assessment can include qualitative assessments of risk (e.g., a “low” risk, an “elevated” risk, a “high” risk; displayed as a particular color (e.g., green indicating a relatively low risk, yellow indicating an “elevated” risk, red indicating a “high” risk)) and/or quantitative assessments of risk (e.g., expressed as a percentage likelihood of contracting the at least one disease, a likelihood score of contracting the at least one disease, etc.). In cases where the assessment is provided with a quantitative measure, one or more computational algorithms can be used to compute the quantitative measure. In some cases, the disease progression information retrieved during the search of the disease database can be used in computations. Moreover, in some cases, providing the user with the assessment comprises providing the user with one or more suggested preventative measures that reduce the rate of progression of the at least on disease in the geographic location. Such preventative measures include seeking immunization against the disease (in the case of pathogenic diseases), taking preemptive medications that inhibit contracting and/or progression of a disease (e.g., immunostimulants such as Vitamin C), avoiding the particular geographic location; wearing personal protective equipment (e.g., gloves, a mask, shoe covers, a hairnet, a respirator, etc.) in the particular geographic location; enhanced personal hygiene measures (e.g., increased frequency of hand washing, increased use of hand sanitizer, etc.).

A graphical user interface (GUI) can be useful in providing the user with the assessment of the risk of contracting the at least one disease. The GUI can be a component of an electronic display of an electronic device, such, as for example, a computer system or other type of electronic device described elsewhere herein. In some cases, an electronic display may include a resistive or capacitive touch screen. The GUI can include one or more graphical elements, such as text, images and/or video. The arrangement of the one or more graphical elements can be tailored to a given output. The arrangement of the one or more graphical elements can be statically or dynamically tailored for the given output.

A GUI can be provided on an electronic display, including the display of a device comprising the computer processor. In some cases, the electronic device is a portable electronic device, as described elsewhere herein. Moreover, a GUI can include textual, graphical and/or audio components. A GUI can be provided on an electronic display, including the display of a device comprising a computer processor. Moreover, in some cases, the assessment is provided via a notification or alert over the network. Such a notification or alert can be provided to an electronic device described herein, including via text message, via email, via social media and/or via an application usable on the electronic device. Moreover, a notification or alert provided to the user may prompt the user to take medical action with respect to the at least one disease.

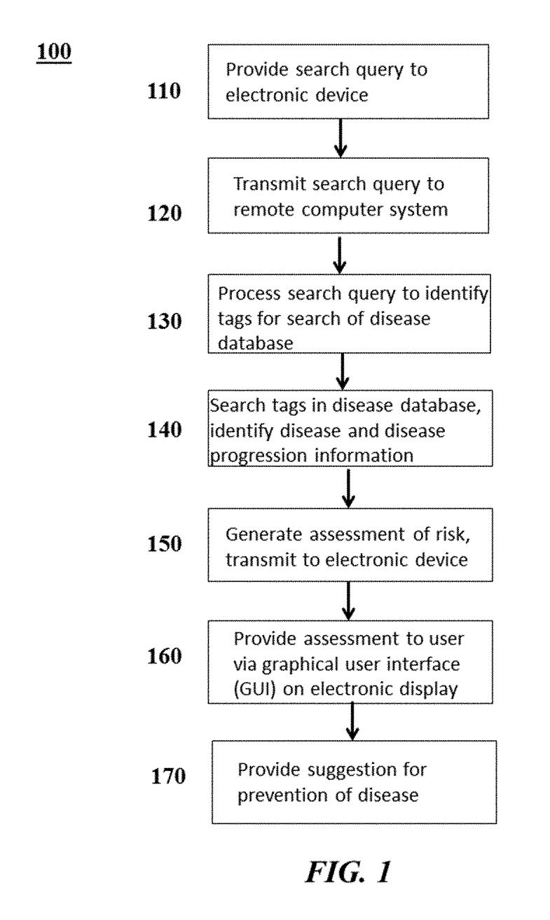

A workflow 100 summarizing an example implementation of the method is shown in FIG. 1. As shown in FIG. 1, a search query is provided 110 by an age 25 user in Beijing who has severe coughing to an electronic device, such as, for example, a smartphone or tablet computer (e.g., via an application installed on the electronic device). The search query contains the terms “severe coughing”, “age 25” and “Beijing, China” and is transmitted 120, via a network (e.g., the Internet), to a remote computer system comprising a computer processor and a disease database as described herein. The remote computer system may be included as part of a decentralized computing network, such as a cloud network. The computer processor processes 130 the search query to identify “severe coughing”, “age 25” and “Beijing” as useful tags to search the disease database and then searches 140 these tags in the disease database. In the disease database, “severe coughing” and “Beijing” are associated with the H1N1 Influenza virus. The disease database comprises disease progression information relating to the increasingly high progression of H1N1 Influenza virus and is associated with subjects in Beijing in the 25-40 years old age group. The search of the disease database identifies 140 the disease as H1N1 Influenza virus and its increasingly high progression within the 25-40 age group in Beijing. A quantitative assessment of the risk of the user contracting H1N1 Influenza is generated 150 by the computer processor and transmitted over the Internet to the user's electronic. The electronic device displays 160 the quantitative assessment on a GUI provided on its display and also displays a qualitative color indicating the relative likelihood of the user contracting H1N1 Influenza. In some cases, the GUI also displays 170 a suggestion to the user that he or she should wash their hands frequently and wear a mask that covers their nose and mouth in order to avoid contracting H1N1 Influenza.

In another aspect, the disclosure provides a method for monitoring at least one disease in a subject. The method includes processing biological samples obtained from the subject at multiple time points to identify one or more biological markers in the biological samples and obtain a quantitative measure of at least a subset of the one or more biological markers across the multiple time points. Each of the one or more biological markers can be indicative of a presence of the at least one disease in the subject. Moreover, the processing can be performed using nucleic acid amplification on each of the biological samples at a sample volume that is less than or equal to about 1 milliliter (mL) and in a time period that is less than or equal to about 10 minutes. The method also includes processing the quantitative measure, with the aid of a computer processor, to determine disease information indicative of a progression or regression of the at least one disease in the subject and generating an output of the disease information. In some cases, the at least one disease is monitored in a fixed geographic location or in a plurality of geographic locations.

In some cases, the disease information is transmitted to a remote data storage unit. The computer processor can be a component of a computer system that is in communication with the remote data storage unit over a network, including any type of network (e.g., decentralized computer network such as a cloud network) described elsewhere herein. Moreover, the remote data storage can comprise any type of data storage medium described elsewhere herein. In some cases, generating the output of the disease information may include provide the disease information to the user on a GUI of an electronic display. The electronic display can be of an electronic device, including a portable electronic device, including a type of electronic device described elsewhere herein.

Additionally, the method can also include providing the subject with a questionnaire to assess a geographic location and/or physiological state of the subject; and identifying the at least one disease from results of the questionnaire. For example, the subject may be asked to provide information regarding one or more physiologic states as described elsewhere herein along with information regarding their current geographic location. The results of the questionnaire can be used to determine the identity of the at least one disease (e.g., based on data regarding diseases associated with the inputted physiologic states and geographic location), which can then in turn be used to determine the disease information. In some cases, the results of a questionnaire can be used to search a disease database and identify the at least one disease and/or disease progression information.

In some cases, the method also includes drawing one or more correlation(s) between the results of the questionnaire and the at least one disease. A non-limiting example of such a correlation includes the prevalence and/or progression or regression of the at least one disease in a subject identifiable by information submitted in the questionnaire. Such a correlation can be useful in assessing the risk a subject identifiable by information submitted in the questionnaire has in contracting the at least one disease. In some cases, a determined correlation is stored in a database for future use and comparison with other analyses of subject biological samples. Additionally, the results of a questionnaire may also be used to guide the selection of target-specific primers used in amplification reactions. Upon using a questionnaire to identify a disease, target-specific primers (e.g., primers that exhibit sequence complementarity to a nucleic acid derived from a pathogenic genome) can be selected for nucleic acid amplification during processing of the biological samples.

Moreover, the questionnaire can be provided to the subject on a user interface (e.g., a GUI) of an electronic device and, in some cases, can be used for machine learning purposes. Questionnaire results can be stored on an electronic device that receives answers to the questionnaire from the user or can be transmitted for storage to a remote data storage unit. Machine learning can aid in future processing of biological samples, processing of quantitative measures, analysis of disease information indicative of a progression or regression of a disease state and can also provide information regarding evaluations across multiple subjects. In some cases, the questionnaire can be provided to the subject on the electronic display of electronic device, including a portable electronic device as described elsewhere herein. In some cases, the questionnaire is provided to the subject via a mobile application (e.g., an “app”).

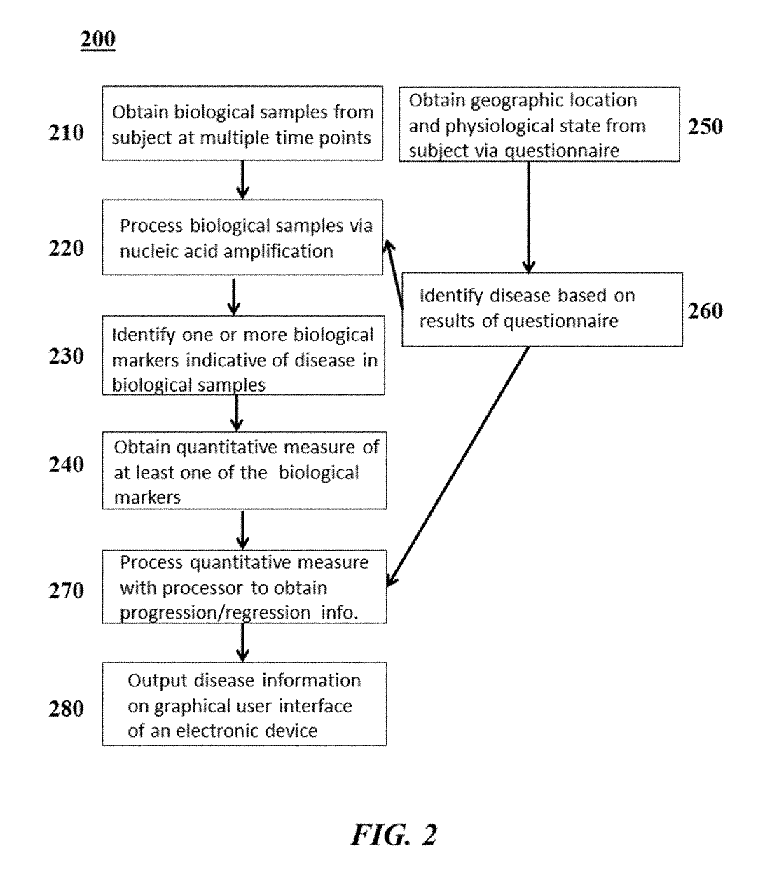

A workflow 200 summarizing an example implementation of the method is shown in FIG. 2. As shown in FIG. 2, biological samples are obtained at multiple time points from a subject 210. The biological samples are provided to a thermocycler in volumes of approximately 0.1 mL and subjected to thermocycling in the presence of amplification reagents (e.g., primers, reverse transcriptase, DNA polymerase, nucleotides, etc.) to reverse transcribe and amplify (e.g., via RT-PCR) nucleic acids (e.g., biological markers) indicative of H1N1 Influenza virus. Nucleic acid amplification is completed in less than 10 minutes. H1N1 Influenza virus specific primers can be used during nucleic acid amplification for targeted amplification of nucleic acids. Amplicons are identified 230 as indicative of H1N1 Influenza virus and the amount of the amplicons generated for each of the biological samples is obtained. In some cases, the amount of amplicons is obtained 240 during amplification, such as via a real time amplification reaction. In parallel or at a different point in time, a questionnaire is provided 250 to the subject via a GUI on an electronic display of an electronic device, such as, for example, a smartphone or tablet computer (e.g., via an application installed on the electronic device). The questionnaire asks the user to provide his or her location along with height, weight and most recent blood pressure reading. The subject enters their location as “Beijing” and provides a height of 1.82 meters (m), a weight of 80 kg and a blood pressure ready of 128 mm Hg systolic/82 mm Hg diastolic. Via a search of a remote disease database, the electronic device identifies 260 H1N1 Influenza virus as a disease associated with the information provided by the subject in the questionnaire. The results of the questionnaire can also be used to select targeted primers for processing 220 of the biological samples via nucleic acid amplification.

Using the amounts of amplicon obtained from the biological samples (e.g., a quantitative measure) and the identified H1N1 Influenza virus information obtained from the questionnaire, the amounts of amplicon obtained from the biological samples are processed 270 with the aid of a computer processor to obtain disease information indicative of progression or regression of H1N1 Influenza virus in the subject. For example, the computer processor may analyze the amplicon data and determine any trend in amount of amplicon over time. An increase in amplicons associated with H1N1 Influenza virus over time may, for example, be indicative of a progression of H1N1 Influenza virus in the subject, whereas a decrease in amplicons associated with H1N1 Influenza virus over time may be indicative of a regression of H1N1 Influenza virus in the subject. Once disease information indicative of progression or regression has been obtained, the disease information is outputted 280 on a GUI of an electronic device, which may be, for example the electronic device used by the subject to provide answers to the questionnaire. In some cases, the disease information is also stored in a memory location of a computer system of a decentralized computing network (e.g., cloud network).

In another aspect, the disclosure provides a method for monitoring at least one disease. The method includes receiving, over a network, disease information for each of a plurality of subjects. For a given subject of the plurality of subjects, the disease information is generated by processing biological samples obtained from the given subject at multiple time points to identify one or more biological markers in the biological samples. Each of the one or more biological markers can be indicative of a presence of the at least one disease in the given subject. The processing can be performed using nucleic acid amplification on each of the biological samples at a sample volume that is less than or equal to about 1 milliliter (mL) and in a time period that is less than about 10 minutes. Furthermore, generating the disease information also includes obtaining a quantitative measure of at least a subset of the one or more biological markers across the multiple time points; and with the aid of a computer processor, processing the quantitative measure to determine the disease information. The disease information is generally indicative of a progression or regression of the at least one disease in the given subject. Moreover, the method also includes compiling the disease information in a memory location and processing the disease information compiled in the memory location to identify a trend of the disease in a given geographic location or across a plurality of geographic locations, followed by generating an output indicative of the trend.

The network can be any suitable network, including a type of network described herein (e.g., the Internet, an internet, an extranet, an intranet, a cloud network, etc.). In some cases, the disease information that is received is transmitted by an electronic device with non-limiting examples of electronic devices described elsewhere herein. The electronic device can be a portable electronic device, including a type of portable electronic device described elsewhere herein.

A trend of the disease in a given geographic location may be with respect to any suitable number of variables and/or considerations. For example, the trend may describe the prevalence rate of the at least one disease over the multiple time points at the geographic location or plurality of geographic locations. In such cases, a positive trend can indicate the progression of the at least one disease at the geographic location or plurality of geographic locations, whereas a negative trend can indicate regression of the at least one disease at the geographic location or plurality of geographic locations. In another example, the trend may describe the prevalence rate of one or more symptoms of the at least one disease over the multiple time points at the geographic location or plurality of geographic locations. In such cases, a positive trend can indicate the progression of symptoms and, thus, the at least one disease, whereas a negative trend can indicate regression of symptoms and, thus, the at least one disease at the geographic location or plurality of geographic locations.

Generating the output indicative of the trend can also include storing the trend in a memory location. Any suitable format of electronic data storage/memory, including those described elsewhere herein, can be used to store the output. In some cases, generating the output indicative of the trend can also include providing the trend to a user on a GUI of an electronic display. The electronic display can be of an electronic device, including a portable electronic device, including an electronic device described elsewhere herein. Moreover, generating the output indicative of the trend can also include providing a notification or alert to a user with respect to the trend. Such a notification of alert can be provided to the user via an electronic device, including a portable electronic device as described elsewhere herein. In some cases, the notification or alert can be provided to a user via text-message, email, via social media, via a mobile application or via any other suitable form of electronic communication. Additionally, in some cases, an output indicative of the trend may comprise providing an update with respect to the trend. The update can be indicative of an increase or a decrease in the prevalence of the at least one disease. An increase or decrease in the prevalence of the at least one disease may be determined by comparing obtained disease information with disease information obtained in a prior analysis.

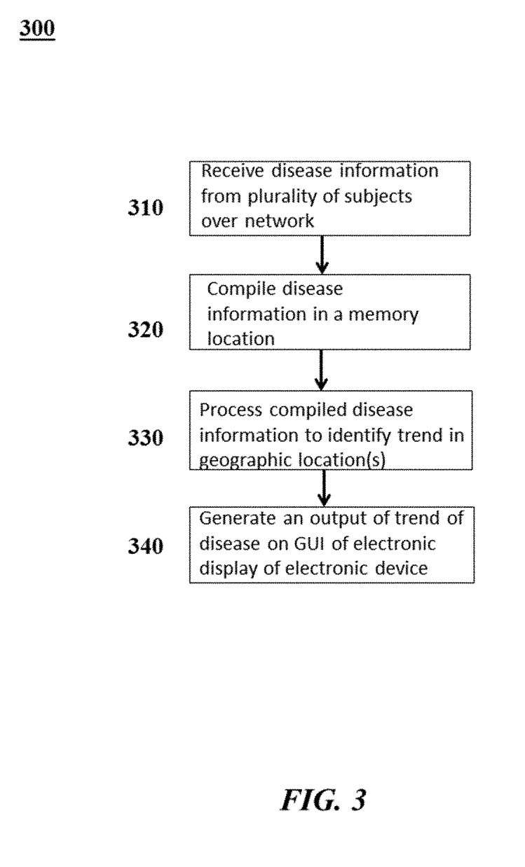

A workflow 300 summarizing an example implementation of the method is shown in FIG. 3. As shown in FIG. 3, H1N1 Influenza virus disease information for each of a plurality of subjects is received 310 by a computer system via a network (e.g., the Internet). The disease information for a given subject of the plurality of subjects is generated by processing samples obtained directly from the given subject at multiple time points. During processing, the biological samples are provided to a thermocycler in volumes of approximately 0.1 mL and subjected to thermocycling in the presence of amplification reagents (e.g., primers, reverse transcriptase, DNA polymerase) to reverse-transcribe and amplify (e.g., via RT-PCR) nucleic acids (e.g., biological markers) indicative of H1N1 Influenza virus. Nucleic acid amplification is completed in less than 10 minutes. H1N1 Influenza virus specific primers can be used during nucleic acid amplification for targeted amplification of nucleic acids. Amplicons are identified as indicative of H1N1 Influenza virus in the subject and the amount of the amplicons generated for each of the biological samples is obtained. In some cases, the amount of amplicons is obtained during amplification, such as via a real time amplification reaction. Moreover, especially in cases where subjects are at geographic locations, processing of the biological samples may be obtained by a designated point-of-care device among a plurality of point-of-care devices.

Using the amounts of amplicon obtained from the biological samples (e.g., a quantitative measure) the amounts of amplicon obtained from the biological samples are processed with the aid of a computer processor to obtain disease information indicative of progression or regression of H1N1 Influenza virus in the given subject. In some cases, the computer processor is a component of an electronic device used to transmit the disease information to the computer system. Moreover, an increase in amplicons associated with H1N1 Influenza virus over time may, for example, be indicative of a progression of H1N1 Influenza virus in the subject, whereas a decrease in amplicons associated with H1N1 Influenza virus over time may be indicative of a regression of H1N1 Influenza virus in the subject.

Once disease information indicative of progression or regression has been obtained, the disease information obtained from the various subjects is compiled 320 into the memory of the computer system. The compiled disease information is then processed 330, perhaps with the aid of a computer processor of the computer system, to identify a trend of H1N1 across Beijing (e.g., a given geographic location) or across cities in China with 1,000,000 or more people (e.g., a plurality of geographic locations). In cases where a disease trend across Beijing is generated, the plurality of subjects may have a geographic location of Beijing. In cases where a disease trend across a plurality of geographic locations is desired, the subjects may be of a given geographic location of the plurality of geographic locations (e.g., a city in China with greater than 1,000,000 people). Following identification of the trend, an output of the trend is generated and displayed to a user on a GUI of an electronic display. The electronic display can be of an electronic device, such as a portable electronic device (e.g., smartphone, tablet computer, etc.) as described elsewhere herein.

The example shown in FIG. 3 can be repeated for any number of cycles to provide an update with respect to the trend. Updated disease information can be processed and provided to the user on the GUI of the electronic device. In some cases, the update may indicate an increase or decrease in the prevalence of H1N1 Influenza in Beijing or cities in China with greater than 1,000,000 people. In order to determine an increase or decrease in prevalence of H1N1 Influenza, processing of updated disease information may include a comparison with disease information obtained from a prior analysis. Such disease information may be compiled and stored in a memory location, including a memory location of the computer system.

Various aspects described herein include the evaluation of disease, including assessments of risk of contracting at least one disease and/or monitoring at least one disease. The at least one disease can be any disease desired for analysis. In some cases, the disease is an infectious disease. In some cases, an infectious disease may be associated with an infectious agent such as a pathogen. Pathogens include both living and non-living species, with non-limiting examples that include a microorganism, a microbe, a virus, a bacterium, an archaeum, a protozoan, a protist, a fungus and a plant. Pathogens can include nucleic acids that may encode, for example, the pathogen's genome. Such nucleic acids can function as biological markers that are indicative of the disease associated with the pathogen. Identification of and quantitation of nucleic acid biological markers can be useful in generating information about the particular disease, including disease progression or regression information as described elsewhere herein.

In some cases, the at least one disease is identifiable by a virus. Non-limiting examples of viruses that can identify an associated disease include human immunodeficiency virus I (HIV I), human immunodeficiency virus II (HIV II), orthomyxovirus, Ebola virus, Dengue virus, influenza viruses (e.g., Influenza A, Influenza B, Influenza C, H1N1 , H2N2, H3N2, H7N7, H1N2, H7N9, H9N2, H7N2, H7N3, H10N7 or H5N1 virus), hepatitis A virus, hepatitis B virus, hepatitis C (e.g., armored RNA-HCV virus) virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox, adenovirus (e.g., Adenovirus Type 55, Adenovirus Type 7), Varicella Zoster virus, Human papilloma virus (HPV), Human T-cell Leukemia Virus (HTLV), mumps virus, Respiratory Syncytial Virus (RSV), parainfluenza virus, Rubella virus Zika virus, Middle East respiratory syndrome (MERS) Virus, Yellow Fever virus, Rift Valley fever virus, Chikungunya Fever virus, enterovirus, Cosksackie virus. Nucleic acids derived from a virus can function as a biological marker that can be identified and quantified.

In some cases, the at least one disease is identifiable by a bacterium. Non-limiting examples of bacteria that can identify an associated disease include Bordetella pertussis, Chlamydia pneumoniae, Chlamydia trachomatis, Campylobacter jejuni, Haemophilus influenzae, Helicobacter pylori, Borrelia bacteria, Mycoplasma pneumoniae, Mycobacterium tuberculosis, Streptococcus pneumoniae, Streptococcus pyogenes, Clostridium tetani, Treponema pallidum, Trypanosoma cruzi, Toxoplasma gondii and Yersinia pestis. Nucleic acids derived from a bacterium can function as a biological marker that can be identified and quantified. In some cases, the least one disease is identifiable by a protozoan. Non-limiting examples of protozoa that can identify an associated disease include Plasmodium and Leishmania donovani. Nucleic acids derived from a protozoan can function as a biological marker that can be identified and quantified.

Moreover, in various aspects of the disclosure, biological samples are obtained from subjects. Any suitable biological sample that comprises nucleic acid may be obtained from a subject. A biological sample may be solid matter (e.g., biological tissue) or may be a fluid (e.g., a biological fluid). Solid samples can be homogenized in a homogenization fluid such that they can be manipulated with fluid handling. In general, a biological fluid can include any fluid associated with a living organism. Non-limiting examples of a biological sample include whole blood (or components of whole blood—e.g., white blood cells, red blood cells, platelets, plasma) obtained from any anatomical location (e.g., tissue, circulatory system, bone marrow) of a subject, cells obtained from any anatomical location of a subject, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, microbiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, and/or other excretions or body tissues.

A biological sample may be obtained from a subject via any suitable route. Non-limiting examples of routes to obtain a biological sample directly from a subject include accessing the circulatory system (e.g., intravenously or intra-arterially via a syringe or other needle), collecting a secreted biological sample (e.g., feces, urine, sputum, saliva, etc.), surgically (e.g., biopsy), swabbing (e.g., buccal swab, oropharyngeal swab), pipetting, and breathing. In some cases, biological samples can be obtained directly from a subject and subsequently processed without subjecting the biological samples to purification to isolate biological markers. For example, where a biological marker is a nucleic acid, the biological samples can be processed without nucleic acid extraction from the biological samples. As another example, the biological samples can be processed without bleaching, sample purification and/or sample extraction.

In some aspects of the disclosure, biological samples are obtained from a subject at multiple time points. Biological samples can be obtained from a subject for any suitable number of time points, depending upon, for example the time period in which monitoring of a disease is desired. For example, a biological sample may be obtained from a subject 2, 3, 4, 5, 6, 7, 8, 9, 10 or more times. Additionally, the time points can be regularly spaced over a period of time (e.g., a daily interval, a weekly interval, a bi-weekly interval, a monthly interval, a quarterly interval, a yearly interval, etc.) or may be irregularly spaced over a period of time. In some cases, the interval selected depends upon the time period in which monitoring of a disease is desired and/or any information that is known about the disease that is being monitored prior to or during sample collection.

In various aspects of the disclosure, biological samples are processed using nucleic acid amplification. Processing of biological samples obtained from a subject can include amplifying nucleic acid biological markers of the biological samples. A nucleic acid biological marker can be a nucleic acid associated with a disease, including a nucleic acid of a pathogen associated with a disease. For example, a nucleic acid biological marker can be a nucleic acid (including nucleic acid of a virus described herein), a bacterial nucleic acid (including nucleic acid of a bacterium described herein) and a protozoan nucleic acid (including nucleic acid of a protozoan described herein).

In various aspects of the disclosure, the amount of biological sample that is processed using nucleic acid amplification can vary depending upon, for example, the availability of biological sample from the subject, the type of nucleic acid amplification used for processing, the capacity of a device (e.g., thermocycler, point-of-care device as described elsewhere herein, etc.) for holding a biological sample for processing. In some cases, relatively small sample sizes may be processed, which can aid in making point-of-care processing feasible and/or minimizing the amount of biological sample needed to obtained from a subject. Minimal requirements for biological sample amount can improve subject compliance by minimizing the time required to obtain a biological sample and/or minimizing any discomfort associated with biological sample acquisition.

As used herein, the amount of a given biological sample that is processed using nucleic acid amplification can be described with sample volume. In general, the volume of biological sample that is processed using nucleic acid amplification is less than or equal to about 1 mL, however can be greater than 1 mL where desired. In some examples, the volume of biological sample that is processed using nucleic acid amplification is less than or equal to about 0.75 mL, is less than or equal to about 0.5 mL, is less than or equal to about 0.25 mL, is less than or equal to about 0.1 mL, is less than or equal to about 0.075 mL, is less than or equal to about 0.050 mL, is less than or equal to about 0.010 mL, is less than or equal to about 0.0075 mL, is less than or equal to about 0.005 mL, is less than or equal to about 0.001 mL or is smaller. In some examples, the volume of biological sample that is processed using nucleic acid amplification is about 0.9 mL, 0.8, mL, 0.7 mL, 0.6 mL, 0.5 mL, 0.4 mL, 0.3 mL, 0.2 mL, 0.1 mL, 0.09 mL, 0.08 mL, 0.07 mL, 0.06 mL, 0.05 mL, 0.04 mL, 0.03 mL, 0.02 mL, 0.01 mL, 0.009 mL, 0.008 mL, 0.007 mL, 0.006 mL, 0.005 mL, 0.004 mL, 0.003 mL, 0.002 mL or 0.001 mL or less.

In various aspects of the disclosure, processing of biological samples can include providing a reaction vessel comprising a given biological sample of the biological samples and reagents necessary for conducting nucleic acid amplification. The given biological sample and reagents can be components in a reaction mixture contained with the reaction vessel. Once provided to the reaction vessel, one or more nucleic acid biological markers of a given biological sample are subjected to nucleic acid amplification under conditions that are sufficient to yield amplification products of the nucleic acid biological markers. As they are at least partial copies of the one or more nucleic acid biological markers, the amplification products are indicative of the presence of the one or more nucleic acid biological markers in the biological sample.

Any suitable reaction vessel may be used for nucleic acid amplification. In some cases, a reaction vessel comprises a body that can include an interior surface, an exterior surface, an open end, and an opposing closed end. Moreover, a reaction vessel may comprise a cap. The cap may be configured to contact the body at its open end, such that when contact is made the open end of the reaction vessel is closed. In some cases, the cap is permanently associated with the reaction vessel such that it remains attached to the reaction vessel in open and closed configurations. In some cases, the cap is removable, such that when the reaction vessel is open, the cap is separated from the reaction vessel. In some cases, a reaction vessel may be sealed, such as hermetically sealed.

A reaction vessel may be of varied size, shape, weight, and configuration. A reaction vessel may be regularly shaped or irregularly shaped. In some examples, a reaction vessel is round, oval tubular, rectangular, square, diamond, circular, elliptical and/or triangular shaped. In some cases, the closed end of a reaction vessel may have a tapered, rounded, or flat surface. Non-limiting examples of types of a reaction vessel include a tube, a well, a capillary tube, a cartridge, a cuvette, a centrifuge tube, or a pipette tip. Reaction vessels may be constructed of any suitable material with non-limiting examples of such materials that include glasses, metals, plastics, and combinations thereof.

In some cases, a reaction vessel is part of an array of reaction vessels. An array of reaction vessels may be particularly useful for automating methods and/or simultaneously processing multiple samples. For example, a reaction vessel may be a well of a microwell plate comprised of a plurality of wells. In another example, a reaction vessel may be held in a well of a thermal block of a thermocycler, where the block of the thermocycler comprises multiple wells each capable of receiving a reaction vessel. An array comprised of reaction vessels may comprise any appropriate number of reaction vessels. For example, an array may comprise at least 2, 4, 6, 8, 10, 12, 14, 16, 18, 20, 25, 35, 48, 96, 144, 384, or more reaction vessels. A reaction vessel part of an array of reaction vessels may also be individually addressable by a fluid handling device, such that the fluid handling device can correctly identify a reaction vessel and dispense appropriate fluid materials into the reaction vessel. Fluid handling devices may be useful in automating the addition of fluid materials to reaction vessels.

In some cases, a reaction vessel may comprise multiple thermal zones. Thermal zones within a reaction vessel may be achieved by exposing different regions of the reaction vessel to different temperature cycling conditions. For example, a reaction vessel may comprise an upper thermal zone and a lower thermal zone. The upper thermal zone may be capable of a receiving a biological sample and reagents necessary to obtain a reaction mixture for nucleic acid amplification. The reaction mixture can then be subjected to a first thermocycling protocol. After a desired number of cycles, for example, the reaction mixture can slowly, but continuously leak from the upper thermal zone to the lower thermal zone. In the lower thermal zone, the reaction mixture is then subjected to a desired number of cycles of a second thermocycling protocol different from that in the upper thermal zone. Such a strategy may be particularly useful when nested PCR is used to amplify nucleic acid. In some cases, thermal zones may be generated within a reaction vessel with the aid of thermal sensitive layering materials within the reaction vessels. In such cases, heating of the thermal sensitive layering materials may be used to release reaction mixtures from one thermal zone to the next. In some cases, the reaction vessel comprises 2, 3, 4, 5, 6, 7, 8, 9, 10, 11, 12, 13, 14, 15 or more thermal zones.

Reagents necessary for nucleic acid amplification include one or more primers having sequence complementarity with one or more nucleic acid biological markers and a polymerizing enzyme that is capable of mediating nucleic acid synthesis in template-directed fashion (e.g., a polymerase). The one or more primers can be directed to DNA biological markers and/or ribonucleic acid (RNA) biological markers, depending upon the particular biological marker(s) under analysis and nucleic acid amplification scheme used. The one or more primers can be designed to target a sequence of a nucleic acid biological marker known to be associated with a disease under study, where amplification of the nucleic acid biological marker via the one or more primers generates amplicons indicative of the presence of the nucleic acid marker in a particular biological sample.

In some cases, reagents necessary for nucleic acid amplification include a polymerase, such as a DNA polymerase. Any suitable DNA polymerase may be used, including commercially available DNA polymerases. Non-limiting examples of DNA polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof.

Any type of nucleic acid amplification reaction may be used to amplify nucleic acid and generate an amplified product. Moreover, amplification of a nucleic acid may linear, exponential, or a combination thereof. Amplification may be emulsion based or may be non-emulsion based. Non-limiting examples of nucleic acid amplification methods include reverse transcription (e.g., reverse transcription PCR (RT-PCR), primer extension, polymerase chain reaction (PCR), ligase chain reaction (LCR), helicase-dependent amplification, asymmetric amplification, rolling circle amplification, and multiple displacement amplification (MDA). In cases where a nucleic acid is deoxyribonucleic acid (DNA) is amplified, any DNA amplification method may be employed. Non-limiting examples of DNA amplification methods include polymerase chain reaction (PCR), variants of PCR (e.g., real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, touchdown PCR), and ligase chain reaction (LCR). In some cases, DNA amplification is linear. In some cases, DNA amplification is exponential. In some cases, DNA amplification is achieved with nested PCR, which can improve sensitivity of detecting amplified DNA products.

In the case of a RNA biological marker, nucleic acid amplification can comprise reverse transcription of the RNA biological marker in parallel with DNA amplification (e.g., RT-PCR nucleic acid amplification), in the presence of a reverse transcriptase (e.g., HIV-1 reverse transcriptase, M-MLV reverse transcriptase, AMV reverse transcriptase, telomerase reverse transcriptase, and variants, modified products and derivatives thereof), DNA polymerase and a primer set for the RNA biological marker. In such a nucleic acid amplification reaction, a RNA primer of the primer and targeted to the RNA biological marker hybridizes with a RNA biological marker and the RNA biological marker is reverse transcribed to DNA product complementary to the RNA via the action of the reverse transcriptase. A second primer of the primer set can then hybridize with the DNA product and be extended via the action of the DNA polymerase to generate a double-stranded DNA product that is indicative of the RNA biological marker in the biological sample. The double-stranded DNA product can then be further amplified, perhaps with additional primers in the primer set, to produce additional double-stranded DNA product. In some cases, parallel reverse transcription and DNA amplification can be performed within a single reaction vessel in a single reaction mixture, without purification and/or removal of the reaction mixture from the reaction vessel. In such cases, the reverse transcriptase, the DNA polymerase, the primer set and a given biological sample can be provided in a single reaction mixture in the reaction vessel.

Nucleic acid amplification can be isothermal or subject to thermocycling. Thermocycling can be performed with the aid of thermocycler. Any suitable thermocycler can be used. In some cases, a thermocycler is a component of a point-of-care device that processes a biological sample obtained from a subject. Moreover, many nucleic acid amplification reactions include one or more primer extension reactions that generate amplified product. During a primer extension reaction, a double-stranded nucleic acid is denatured into single-strands (if necessary), a primer hybridized is to one or both of the single-strands and the primer is extended via the action of a polymerizing enzyme (e.g., a DNA polymerase, a reverse transcriptase) in template-directed fashion. Primer extension reactions can include a cycle of incubating nucleic acids to be amplified at a denaturation temperature for a denaturation duration and incubating the nucleic acids to be amplified at an elongation temperature for an elongation duration.

Denaturation temperatures may vary depending upon, for example, the particular biological sample processed, the particular nucleic acid biological markers under analysis in the biological sample, the reagents used, and/or the desired reaction conditions. For example, a denaturation temperature may be from about 80° C. to about 110° C. In some examples, a denaturation temperature may be from about 90° C. to about 100° C. In some examples, a denaturation temperature may be from about 90° C. to about 97° C. In some examples, a denaturation temperature may be from about 92° C. to about 95° C. In still other examples, a denaturation temperature may be about 80°, 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

Denaturation durations may vary depending upon, for example, the particular biological sample processed, the particular nucleic acid biological markers under analysis in the biological sample, the reagents used, and/or the desired reaction conditions. For example, a denaturation duration may be less than or equal to about 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. For example, a denaturation duration may be no more than 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second.

Elongation temperatures may vary depending upon, for example, the particular biological sample processed, the particular nucleic acid biological markers under analysis in the biological sample, the reagents used, and/or the desired reaction conditions. For example, an elongation temperature may be from about 30° C. to about 80° C. In some examples, an elongation temperature may be from about 35° C. to about 72° C. In some examples, an elongation temperature may be from about 45° C. to about 65° C. In some examples, an elongation temperature may be from about 35° C. to about 65° C. In some examples, an elongation temperature may be from about 40° C. to about 60° C. In some examples, an elongation temperature may be from about 50° C. to about 60° C. In still other examples, an elongation temperature may be about 35°, 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C.

Elongation durations may vary depending upon, for example, the particular biological sample processed, the particular nucleic acid biological markers under analysis in the biological sample, the reagents used, and/or the desired reaction conditions. For example, an elongation duration may be less than or equal to 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. For example, an elongation duration may be no more than 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second.

In some aspects of the disclosure, a biological sample can be subjected to multiple cycles of a primer extension reaction can be conducted. Any suitable number of cycles may be conducted. For example, the number of cycles conducted may be less than about 100, 90, 80, 70, 60, 50, 40, 30, 20, 10, or 5 cycles. The number of cycles conducted may depend upon, for example, the number of cycles (e.g., cycle threshold value (Ct)) necessary to obtain a detectable amplified product. For example, the number of cycles necessary to obtain a detectable amplified product may be less than about or about 100 cycles, 75 cycles, 70 cycles, 65 cycles, 60 cycles, 55 cycles, 50 cycles, 40 cycles, 35 cycles, 30 cycles, 25 cycles, 20 cycles, 15 cycles, 10 cycles, or 5 cycles. Moreover, in some cases, a detectable amount of an amplifiable product may be obtained at a cycle threshold value (Ct) of less than 100, 75, 70, 65, 60, 55, 50, 45, 40, 35, 30, 25, 20, 15, 10, or 5.

In some cases, a biological sample may be subjected to a plurality of series of primer extension reactions. An individual series of the plurality may comprise multiple cycles of a particular primer extension reaction, characterized, for example, by particular denaturation and elongation conditions as described elsewhere herein. Generally, each individual series differs from at least one other individual series in the plurality with respect to, for example, a denaturation condition and/or elongation condition. An individual series may differ from another individual series in a plurality of series, for example, with respect to any one, two, three, or all four of denaturing temperature, denaturing duration, elongation temperature, and elongation duration. Moreover, a plurality of series may comprise any number of individual series such as, for example, at least about or about 2, 3, 4, 5, 6, 7, 8, 9, 10, or more individual series.

For example, a plurality of series of primer extension reactions may comprise a first series and a second series. The first series, for example, may comprise more than ten cycles of a primer extension reaction, where each cycle of the first series comprises (i) incubating a reaction mixture at about 92° C. to about 95° C. for no more than 30 seconds followed by (ii) incubating the reaction mixture at about 35° C. to about 65° C. for no more than about one minute. The second series, for example, may comprise more than ten cycles of a primer extension reaction, where each cycle of the second series comprises (i) incubating the reaction mixture at about 92° C. to about 95° C. for no more than 30 seconds followed by (ii) incubating the reaction mixture at about 40° C. to about 60° C. for no more than about 1 minute. In this particular example, the first and second series differ in their elongation temperature condition. The example, however, is not meant to be limiting as any combination of different elongation and denaturing conditions could be used.

An advantage of conducting a plurality of series of primer extension reaction may be that, when compared to a single series of primer extension reactions under comparable denaturing and elongation conditions, the plurality of series approach yields a detectable amount of amplified product that is indicative of the presence of a nucleic acid biological marker in a biological sample with a lower cycle threshold value. Use of a plurality of series of primer extension reactions may reduce such cycle threshold values by at least about or about 1%, 5%, 10%, 15%, 20%, 25%, 30%, 35%, 40%, 45%, 50%, 55%, 60%, 65%, 70%, 75%, 80%, 85%, 90%, or 95% when compared to a single series under comparable denaturing and elongation conditions.

Moreover, a biological sample may be preheated prior to conducting a primer extension reaction. The temperature (e.g., a preheating temperature) at which and duration (e.g., a preheating duration) for which a biological sample is preheated may vary depending upon, for example, the particular biological sample being analyzed. In some examples, a biological sample may be preheated for no more than about 60 minutes, 50 minutes, 40 minutes, 30 minutes, 25 minutes, 20 minutes, 15 minutes, 10 minutes, 9 minutes, 8 minutes, 7 minutes, 6 minutes, 5 minutes, 4 minutes, 3 minutes, 2 minutes, 1 minute, 45 seconds, 30 seconds, 20 seconds, 15 seconds, 10 seconds, or 5 seconds. In some examples, a biological sample may be preheated at a temperature from about 80° C. to about 110° C. In some examples, a biological sample may be preheated at a temperature from about 90° C. to about 100° C. In some examples, a biological sample may be preheated at a temperature from about 90° C. to about 97° C. In some examples, a biological sample may be preheated at a temperature from about 92° C. to about 95° C. In still other examples, a biological sample may be preheated at a temperature of about or at least about 80°, 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

In various aspects that include processing of biological samples with nucleic acid amplification, the time required to complete processing vary depending upon, for example, the amount of biological sample to be processed, the capabilities of a device used for processing, and the amount of any biological marker present in the sample(s). In general, processing of a biological sample(s) with nucleic acid amplification is achieved in less than or equal to about 10 min., however can take longer depending upon the particular processing strategy. In some examples, processing of a biological sample(s) with nucleic acid amplification is achieved in about 0.1 min. to about 10 min. In some examples, processing of a biological sample(s) with nucleic acid amplification is achieved in about 0.5 min. to about 10 min. In some examples, processing of a biological sample(s) with nucleic acid amplification is achieved in about 1 min. to about 10 min. In some examples, processing of a biological sample(s) with nucleic acid amplification is achieved in about 0.5 min to about 5 min. In some examples, processing of a biological sample(s) with nucleic acid amplification is achieved in less than or equal to about 9 min., less than or equal to about 8 min., less than or equal to about 7 min., less than or equal to about 6 min., less than or equal to about 5 min., less than or equal to about 4 min., less than or equal to about 3 min., less than or equal to about 2 min., less than or equal to about 1 min., less than or equal to about 0.75 min., less than or equal to about 0.5 min., less than or equal to about 0.1 min. or less.

As described elsewhere herein, various aspects of the disclosure include obtaining a quantitative measure of one or more biological markers across multiple time points. A quantitative measure can include an absolute amount (e.g., mass, mole amount, volume, concentration) and/or a relative amount (e.g., relative mass (e.g., mass percentage, mole percentage, volume percentage) of a biological marker in a biological sample. In some cases, a quantitative measure may include a set of values (e.g., a set of amounts across the multiple time points analyzed). Moreover, as is also described elsewhere herein with respect to various aspects, a quantitative measure is processed to determine disease information, including disease information that is indicative of a progression or regression of a disease. Any desired type of processing can be completed. Processing may include, for example, comparing quantitative measures at multiple time points to a reference to identify progression or regression of a disease in a subject. Such a reference can comprise an amount or relative amount of a biological marker associated with a healthy state (e.g., where a disease is not present) and/or at a differing time point than a time point of the multiple time points analyzed. In some cases, a comparison can be made between quantitative measures across the multiple time points, which can be used to determine progression or regression of a disease over the multiple time points analyzed. Comparisons between multiple time points analyzed can be useful in generating updates to trends obtained from processing of disease information indicative of progression or regression of disease.

Additional reagents can be added to an amplification reaction mixture to aid in providing a quantitative measure of a nucleic acid biological marker in a biological sample being processed. In some cases, such reagents include a reporter agent that yields a detectable signal whose presence or absence is indicative of the presence of an amplified product and, thus, a given nucleic acid biological marker in the biological sample analyzed. The intensity of the detectable signal may be proportional to the amount of amplified product and, thus, the amount of nucleic acid biological marker in a given biological sample. For example, a RNA biological marker is processed via parallel reverse transcription and amplification of the DNA obtained from reverse transcription, reagents necessary for both reactions may be included in an amplification reaction mixture and may also comprise a reporter agent may yield a detectable signal that is indicative of the presence of the amplified DNA product and, thus, the RNA biological marker. In some cases, a reporter agent enables real-time amplification methods that can be used to obtain a quantitative measure during nucleic acid amplification, including real-time PCR for DNA amplification.

Reporter agents may be linked with nucleic acids, including amplified products, covalently or non-covalently. Non-limiting examples of non-covalent linkages include ionic interactions, Van der Waals forces, hydrophobic interactions, hydrogen bonding, and combinations thereof. In some cases, reporter agents may bind to initial reactants and changes in reporter agent levels may be used to detect amplified product. In some cases, reporter agents may only be detectable (or non-detectable) as nucleic acid amplification progresses. In some cases, an optically-active dye (e.g., a fluorescent dye) may be used as may be used as a reporter agent. Non-limiting examples of dyes include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and −2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, −41, −42, −43, −44, −45 (blue), SYTO-13, −16, −24, −21, −23, −12, −11, −20, −22, −15, −14, −25 (green), SYTO-81, −80, −82, −83, −84, −85 (orange), SYTO-64, −17, −59, −61, −62, −60, −63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5-(or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores.

In some cases, a reporter agent may be a sequence-specific oligonucleotide probe that is optically active when hybridized with an amplified product. Due to sequence-specific binding of the probe to the amplified product, use of oligonucleotide probes can increase specificity and sensitivity of detection. A probe may be linked to any of the optically-active reporter agents (e.g., dyes) described herein and may also include a quencher capable of blocking the optical activity of an associated dye. Non-limiting examples of probes that may be useful used as reporter agents include TaqMan probes, TaqMan Tamara probes, TaqMan MGB probes, or Lion probes.

In some cases and where a reporter agent may be an RNA oliognucleotide probe that includes an optically-active dye (e.g., fluorescent dye) and a quencher positioned adjacently on the probe. The close proximity of the dye with the quencher can block the optical activity of the dye. The probe may bind to a target sequence to be amplified. Upon the breakdown of the probe (e.g., with the exonuclease activity of a DNA polymerase) during amplification, the quencher and dye are separated, and the free dye regains its optical activity that can subsequently be detected.

In some cases, a reporter agent may be a molecular beacon. A molecular beacon includes, for example, a quencher linked at one end of an oligonucleotide in a hairpin conformation. At the other end of the oligonucleotide is an optically active dye, such as, for example, a fluorescent dye. In the hairpin configuration, the optically-active dye and quencher are brought in close enough proximity such that the quencher is capable of blocking the optical activity of the dye. Upon hybridizing with amplified product, however, the oligonucleotide assumes a linear conformation and hybridizes with a target sequence on the amplified product. Linearization of the oligonucleotide results in separation of the optically-active dye and quencher, such that the optical activity is restored and can be detected. The sequence specificity of the molecular beacon for a target sequence on the amplified product can improve specificity and sensitivity of detection.

In some cases, a reporter agent may be a radioactive species. Non-limiting examples of radioactive species include ¹⁴C, ¹²³I, ¹²⁴I, ¹²⁵I, ¹³¹I, Tc99m, ³⁵S, or ³H.

In some cases, a reporter agent may be an enzyme that is capable of generating a detectable signal. Detectable signal may be produced by activity of the enzyme with its substrate or a particular substrate in the case the enzyme has multiple substrates. Non-limiting examples of enzymes that may be used as reporter agents include alkaline phosphatase, horseradish peroxidase, I²-galactosidase, alkaline phosphatase, β-galactosidase, acetylcholinesterase, and luciferase.

Detection of amplified product via a reported agent may be accomplished with any suitable detection modality. The particular type of detection method used may depend, for example, on the particular amplified product, the type of reaction vessel used for amplification, other reagents in a reaction mixture, and the particular type of reporter agent use. Non-limiting examples of detection methods include optical detection, spectroscopic detection, electrostatic detection, electrochemical detection, and the like. Optical detection methods include, but are not limited to, fluorimetry and UV-vis light absorbance. Spectroscopic detection methods include, but are not limited to, mass spectrometry, nuclear magnetic resonance (NMR) spectroscopy, and infrared spectroscopy. Electrostatic detection methods include, but are not limited to, gel based techniques, such as, for example, gel electrophoresis. Electrochemical detection methods include, but are not limited to, electrochemical detection of amplified product after high-performance liquid chromatography separation of the amplified products.

In various aspects of the disclosure, information, such as a trend, a quantitative measure of a biological marker in a biological sample, disease information and/or updates or alerts thereof is provided to a user. As described elsewhere herein, information can be provided to a user via a GUI of an electronic display of an electronic device. In some cases, the user is a subject from which biological samples are obtained and analyzed. In other cases, the user can be a healthcare professional. Non-limiting examples of health-care professionals include medical personnel, clinicians (e.g., doctors, nurse practitioners (PACs), nurses, medical assistants, physical therapists, medical interns, medical technicians), laboratory personnel (e.g., hospital laboratory technicians, research scientists, pharmaceutical scientists), a clinical monitor for a clinical trial, an employee of a hospital or health system, an employee of a health insurance company, an employee of a pharmaceutical company, a public health worker, a humanitarian aid worker, or others in the health care industry. In some cases, the GUI can be a GUI of an application run by the electronic device. Where the electronic device is a portable device (e.g., a smartphone, a portable music player, a tablet computer, etc.), the application may be a mobile application (an “app”) that can be run on the portable device. Mobile applications include software that is designed to be run on and/or displayed on a mobile device.

Moreover, in some cases, information provided to a user may be provided in a report that can be displayed by a user interface, such as GUI (including a GUI of a mobile application) of an electronic device. Such a report can include any number of desired elements, with non-limiting examples that include information regarding a subject (e.g., sex, age, race, health status, etc.), raw data, processed data (e.g. graphical displays (e.g., figures, charts, data tables, data summaries), quantitative measures, disease information, correlations between disease information and results of a questionnaire, disease trend information, diagnosis information, prognosis information, recommendations for future action, recommendations for treatment of a disease, recommendations for prevention of a disease, and combinations thereof. Additionally, reports may be stored in an electronic database, such as a disease database, such that they are accessible for comparison with future reports.

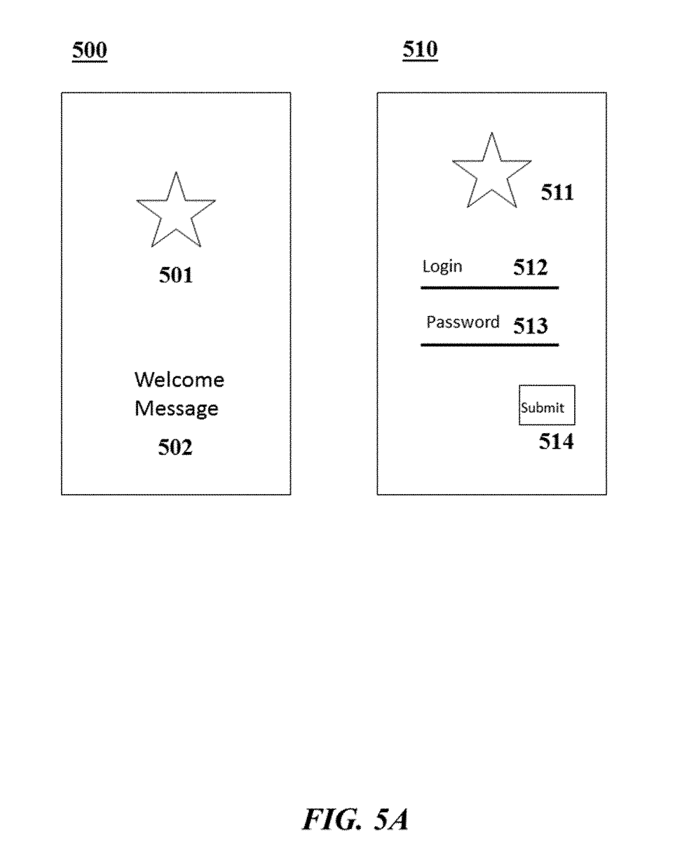

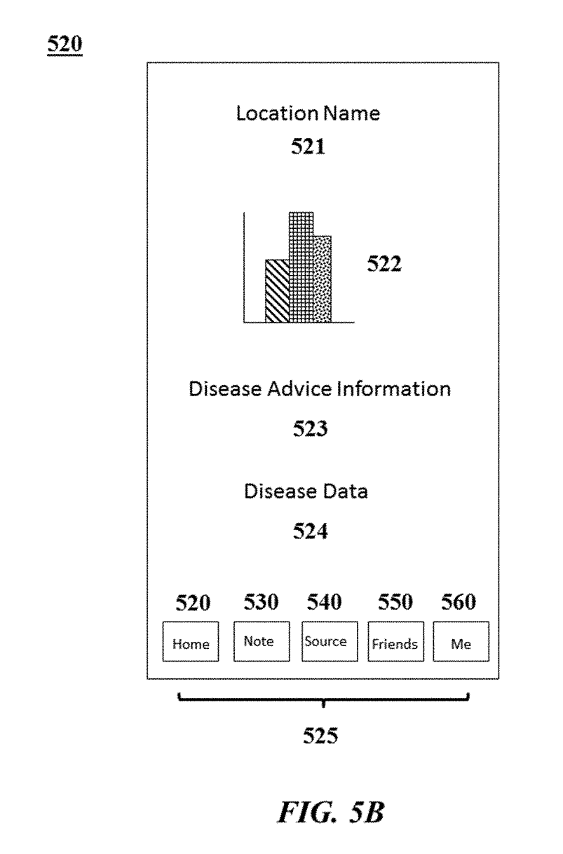

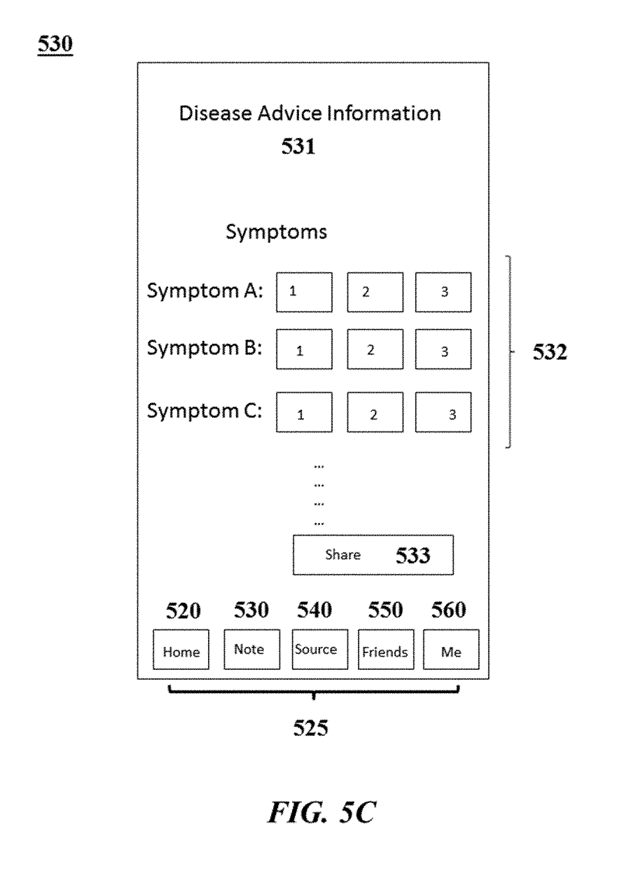

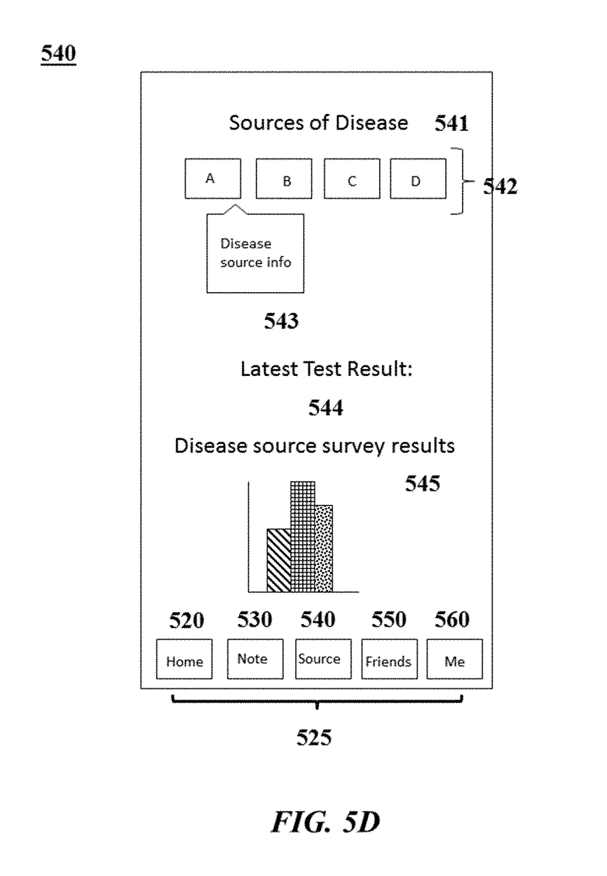

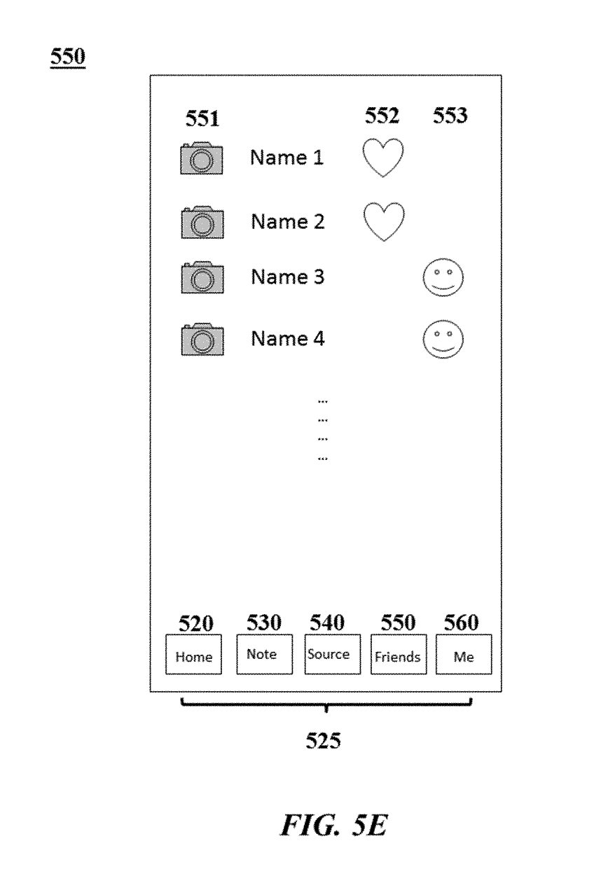

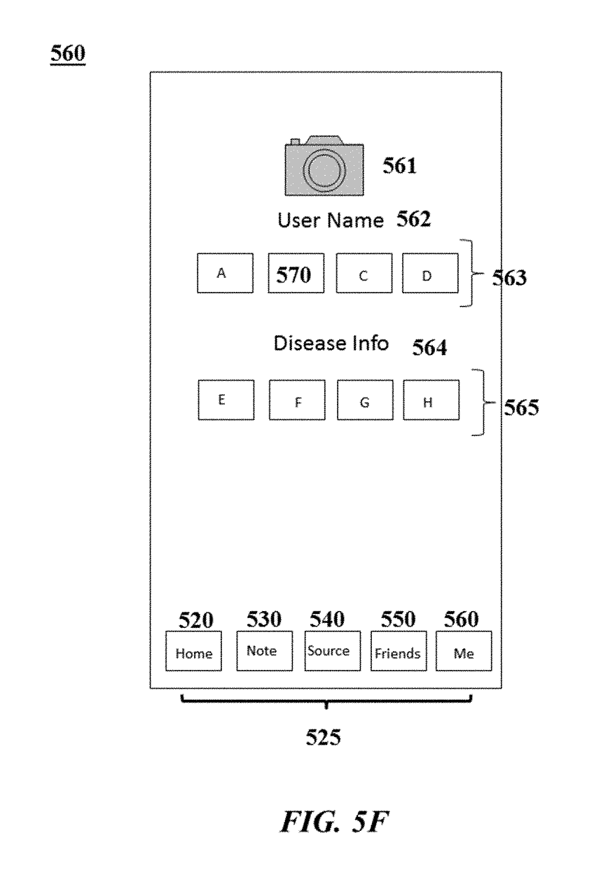

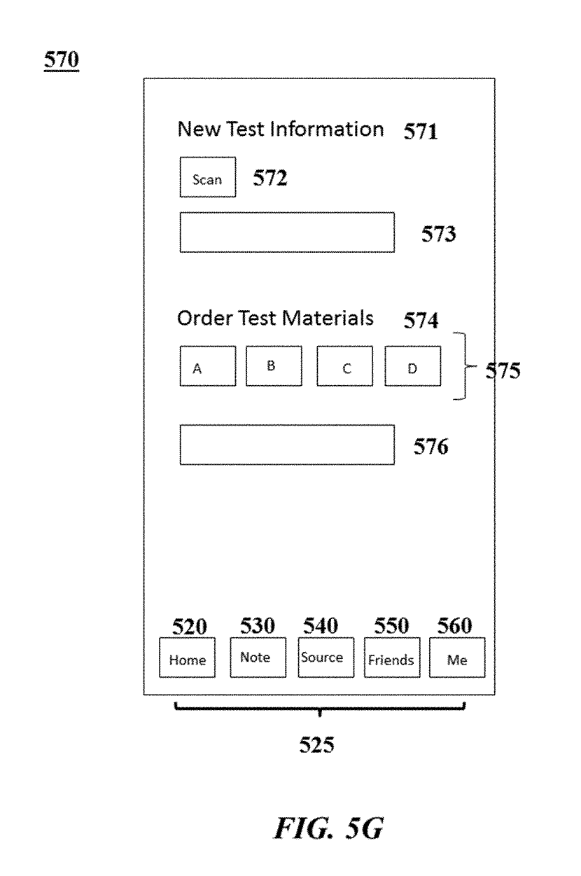

An example mobile application running an electronic device having a touchscreen and that can aid in practicing various aspects of the disclosure is schematically depicted in FIGS. 5A-5G. With reference to FIG. 5A, the application (e.g., mobile application) can provide a welcome screen 500 upon execution of the mobile application. The welcome screen 500 can include one or more graphical elements 501 (e.g., company logo, user photograph, etc.) and/or a welcome message 502 (e.g., the application name, a user welcome, a slogan, a trademark, etc.). Following display of the welcome screen 500, the application then displays a login screen 510 that can include one or more graphical elements 511 along with entry fields for a login 512 (e.g., username, email address, or other identification string) and password 513. Upon entry of the login 512 and password 513 information by the user, the user taps a submit button 514 to enter the application.

Upon entry of the appropriate login 512 and password 513 to login screen 510, the application then displays a home screen 520 that is schematically depicted in FIG. 5B. The home screen 520 can include a location name 521 that can be entered by the user into an entry field (not shown) or may be obtained automatically via GPS capabilities of the electronic device running the mobile application. The home screen 520 can also include a graphical summary 522 of disease data (e.g., temperature at the location, temperature difference from a different location, prevalence of disease at the location, PM2.5 levels at the location, weather information, etc.). A more comprehensive numerical display 524 of the disease data summarized in the graphical summary 522 can also be provided. Based on the disease data summarized on the home screen 522, and/or any other data, the application generates or retrieves disease advice information 523 that is presented to the user. The disease advice information can include suggested disease treatment and/or prevention measures for the user to take. Moreover, the home screen also includes a navigation section 525 that includes graphical buttons (520, 530, 540, 550 and 560 corresponding to screens 520, 530, 540, 550 and 560 as described herein) that each route the user to another screen within the mobile application.

Upon tapping button 530 of navigation section 525, the mobile application displays a note intake screen 530 that is schematically depicted in FIG. 5C. On note intake screen 530, the user is presented with a variety of symptoms (e.g., “Symptom A”, “Symptom B”, “Symptom C”) with option buttons 532 for each symptom. While only three symptom options are shown in FIG. 5C, any number of relevant symptoms can be presented to the user. For each symptom, the user selects the appropriate button (“1”, “2”, or “3” buttons next to each symptom). For example, Symptom A may be hourly sneezing rate (where each button next to Symptom A represents a hourly sneezing rate), Symptom B may be pain location (where each button next to Symptom B represents a pain location/type (e.g., headache, sore throat, everywhere, etc.)) and Symptom C may be body temperature (where each button next to Symptom C represents a particular body temperature). Upon entering appropriate symptom information into note intake screen 530, the mobile application processes the symptom information and provides disease advice information 531. Disease advice information 531 can be populated as disease advice information 523 in home screen 520. Furthermore, note intake screen 530 can also include a button 533 that a user can tap to share entered symptom information on social media. Moreover, note intake screen 530 can also include navigation section 525.

Upon tapping button 540 of navigation section 525, the mobile application displays a disease source screen 540 that is schematically depicted in FIG. 5D. On disease source screen 540, the user is presented with buttons 542 (“A”, “B”, “C”, “D”) each having a possible source 541 of the one or more disease. Where only four buttons are shown in FIG. 5D, any appropriate number of buttons may be displayed. Upon tapping a button, the user is presented with a box 543 that provides more information about the source of the disease. For example, button “A” of the buttons 542 may correspond to a sink. Upon tapping button “A”, the user is presented with box 543 with more details on how a sink could be a source of disease (e.g., disease infection). Moreover, screen 540 also can also include a latest test result 544 from testing of a disease source (e.g., via processing of samples obtained from a particular source) and/or survey results 545 provided by users of the mobile application as to what sources that they have detected disease. In addition, disease source screen 540 can also include navigation section 525.

Upon tapping button 550 of navigation section 525, the mobile application displays a social media screen 550 that is schematically depicted in FIG. 5E. Social media screen 550 displays various other users of the mobile application that the user has added to a “friends” list. For each added user, a photograph or other avatar 551 is displayed along with the user name (e.g., “Name 1”, “Name 2”, “Name 3” and “Name 4”). Each added user entry can also include a “comfort” button 552 and/or a “like” button 553. Where the mobile application recognizes that an added user likely has a disease, the mobile application user can tap the “comfort” button 552 to send the added user a message regarding their disease (e.g., a get well message, a comfort message, etc.). Where the mobile application recognizes that an added user is likely healthy, the mobile application user can tap the “like” button 553 to acknowledge the added user's positive physiological state. Social media screen 550 can include any number of added users and may be displayed over several pages (e.g., accessible by swiping the screen or tapping a navigation button). Moreover, social media screen 550 can also include navigation section 525.

Upon tapping button 560 of navigation section 525, the mobile application displays a user information screen 560 that is schematically depicted in FIG. 5F. User information screen 560 can include a photograph or other avatar 561 that is provided by the user and can be used in social media on other user's social media screens. User information screen 560 can also display the user's name 562. User information buttons 563 can also be displayed (buttons “A”, 570, “C” and “D”). These buttons can be used to access a variety of screens including accessing history of personal disease monitoring (e.g., as described elsewhere herein), access history of note intake, access messages received from other users via social media (e.g., comfort messages, like messages as described above with respect to social media screen 550), reviewing and editing user information (e.g., name, avatar, location, sex, age, physiological information, etc.), and also to access information for obtaining disease monitoring materials. User information screen 560 can also include disease information buttons 564 that each provide the user with access to information about a disease or group of diseases. Buttons 564 can also include a button to view the prevalence of a particular disease or grouping of diseases in a plurality of geographic locations and/or world-wide. Furthermore, user information screen 560 can also include navigation section 525.

Upon tapping button 570 of user information screen 560, the mobile application displays a test information screen 570 that is schematically depicted in FIG. 5G. Test information screen 570 can include a new test information section 571 that permits the user to associate disease monitoring tests with their profile. This section can include a “scan” button 572 that accesses an electronic device's camera (if present) and recognizes a barcode imaged with the camera and associated with materials (e.g., consumables) associated with disease monitoring. As an alternative to scanning, the section also includes an input field 573 where a user can enter in a barcode or other type of identifying information. Moreover, test information screen can also function as an order form for materials necessary for conducting disease monitoring. In such cases, the mobile application can display a materials ordering section 574, whereby the user is presented with buttons 575 that each represent an address previously associated with the user. Upon tapping the appropriate address, the user can finalize the order in an additional screen (not shown). Alternatively, address information can be entered into a field 576 and then further processed. Furthermore, test information screen 570 can also include navigation section 525.

A point-of-care device as used herein generally refers to a device that is suitable for function at or near a location at which a biological sample is obtained from a subject. Point-of-care devices can be portable and/or capable of being moved to near or at a location of a subject. Moreover, a point-of-care device can be capable of processing a biological sample and/or obtaining one or more quantitative measures of biological markers. Data from the point-of-care device can be analyzed by a computer processor on the point-of-care device or may be transmitted, over a network, to a remote computer system that receives the data and further processes it (e.g., generates a quantitative measure of one or more biological markers, determines disease information, determines a trend, etc.). The processed data can be sent, over a network, back to the point-of-care device or to a different electronic device to be displayed to the user. Furthermore, in some aspects of the disclosure, including those that include obtaining biological samples from a plurality of subjects, biological samples from a given subject may be processed at a designated point-of-care device among a plurality of point-of-care devices. For example, monitoring of a disease may include monitoring the disease across subjects in a plurality of geographic locations. At a given geographic location of the plurality of geographic locations, a point-of-care device may be used to process biological samples obtained from subject(s) at the given geographic locations.

Moreover, a point-of-care device can include a reaction vessel that can receive a biological sample from a subject and any reagents necessary for nucleic acid amplification. A point-of-care device can also include a heater and/or a cooling system in order to modulate temperature during nucleic acid amplification. Additionally, a point-of-care device can include a detector that detects signals indicative of biological markers in the biological samples. Such signals can be useful in providing a quantitative measure of a biological marker in the sample. The detector and its modality of detection can be any suitable detector/detection modality, including types of detectors described elsewhere herein. In some cases, a point-of-care device may include on-board circuitry and/or computer processor that can be used to receive data, over a network, from a remote computer system and/or process a quantitative measure, process disease information, generate trends, provide updates, provide alerts/notifications.

In some aspects, the present disclosure involves providing a user with an assessment of a risk of contracting at least one disease while travelling and/or optimizing an itinerary.

In one aspect among the some aspects, the present disclosure provides a method for providing a user with an assessment of a risk of contracting at least one disease. The method may comprise receiving, over a network, a search query of a user, which search query may include information related to a destination, and optionally one or more waypoints. With the aid of a computer processor, the search query may be processed to identify one or more geographic location tags associated with the destination and optionally the one or more waypoints for searching in a disease database. The disease database may comprise disease progression information that is indicative of a progression or regression of the at least one disease in one or more geographic locations. The one or more geographic locations may include the destination. The method may further comprise searching the disease database using the one or more geographic location tags to identify the at least one disease and the disease progression information. The method may further comprise, providing the user with the assessment of the risk of contracting the at least one disease at the destination and, in some cases, the one or more waypoints, based on the identified disease progression information.

The term “destination”, as used herein, refers to a geographic location that the user as described in the present disclosure travels to or plans to travel to. The destination may be a geographic location as described elsewhere herein. Alternatively or additionally, the destination may be an entity associated with a geographic location as described elsewhere herein. For example, the destination may be a building, a business location (such as a restaurant, a retail store, a department store, a shopping mall, an office building, a bank, etc.), a tourist site, a public facility, a transportation hub (such as a train station, an airport, a coach station, a ferry, etc.), and the like, as long as such a destination may be associated with a geographic location as described elsewhere herein. A destination is associated with a geographic location if it can be recognized manually or automatically as located in a geographic location or its relative position to a geographic location can be determined manually or automatically. In some embodiments, with the aid of a computer processor, a destination may be associated with a geographic location tag which may be used to search a disease database.

The term “waypoint”, as used herein, refers to transient destinations where a passenger may stop over before moving to the next or final destination. All limitations on the destination may be applicable to the waypoint. For example, a waypoint is associated with a geographic location if it can be recognized manually or automatically as located in a geographic location or its relative position to a geographic location can be determined manually or automatically. Although the term “transient” is used in defining the waypoint, it should not be construed as particular limitation on the duration of stopover that the passenger stays at the waypoint. For example, the passenger may stay at the waypoint for less than 10 minutes, 10 minutes, 20 minutes, 30 minutes, 1 hour, 2 hours, 3 hours, 5 hours, 12 hours, 1 days, 2 days, 5 days, or more than 5 days, or any duration between these values. In some embodiments, with the aid of a computer processor, a waypoint may be associated with a geographic location tag which may be used to search a disease database. In some embodiments, reference to one or more waypoints includes reference to the starting point and/or the destination.

The search query of the user can be provided to an electronic device that transmits the search query over the network for processing by the computer processor as described elsewhere herein. Additionally, the computer processor can be a component of a remote computer system networked with the electronic device. The network may be a network as described elsewhere herein, such as the Internet, an internet and/or extranet, an intranet and/or extranet that is in communication with the Internet, a cellular phone network that is in communication with the Internet, or a network “cloud”.

The disease database may be any disease database as described elsewhere herein that includes disease progression information as described elsewhere herein. The disease progression information is indicative of a progression or regression of the at least one disease in one or more geographic locations. As described above, such information may include an incidence rate, a longitudinal incidence rate, a mortality rate, a longitudinal mortality rate and/or the prevalence of one or more symptoms associated with the at least one disease in the one or more geographic locations.

In some embodiments, the user may be provided with the assessment of the risk of contracting the at least one disease on a graphical user interface (GUI) as described elsewhere herein. For example, the GUI may be a component of an electronic display of an electronic device as described elsewhere herein. In some embodiment, the electronic device may be a portable electronic device. In some embodiment, the graphical user interface may be provided by a mobile computer application.

In some embodiment, the search query may further include an identity and/or physiological state of the user. The identity and physiological state may be any identity and physiological state as described elsewhere herein. For example, the identity may include at least one of a name, age and sex of the user; the physiological state may include at least one of a heart rate, blood pressure, coughing frequency, coughing intensity, sneezing frequency, sneezing intensity, a level of chest congestion, a level of nasal congestion, body temperature, sweat level, weight, height, breathing rate, blood pressure, nerve conduction velocity, lung capacity, urine production rate, defecation rate, the presence of enlarged lymph nodes and a biochemical profile of a bodily fluid of the user.

In some embodiment, the search query may include a starting point of the user. The term “starting point”, as used herein, refers to a geographic location that the user as described in the present disclosure starts or plans to start the travel at. The starting point may be a geographic location as described elsewhere herein. Alternatively or additionally, the starting point may be an entity associated with a geographic location as described elsewhere herein. For example, the destination may be a building, a business location (such as a restaurant, a retail store, a department store, a shopping mall, an office building, a bank, etc.), a tourist site, a public facility, a transportation hub (such as a train station, an airport, a coach station, a ferry, etc.), and the like, as long as such a starting point may be associated with a geographic location as described elsewhere herein. A starting point is associated with a geographic location if it can be recognized manually or automatically as located in a geographic location or its relative position to a geographic location can be determined manually or automatically. In some embodiments, with the aid of a computer processor, a starting point may be associated with a geographic location tag which may be used to search a disease database.

Alternatively, the starting point may be automatically determined by an electronic device via, for example, the capability for accessing a global navigation satellite system, such as the global positioning system (GPS) system, the Globalnaya navigatsionnaya sputnikovaya Sistema (GLONASS), Indian Regional Navigation Satellite System (IRNSS), BeiDou Navigation Satellite System (BDS), Galileo (the European satellite navigation system), and the like. The electronic device may be any electronic as described elsewhere herein. For example, the electronic device may be a personal computer, a portable electronic device (such as a mobile telephone), a tablet computer, or the like.

Alternatively, the starting point may be determined automatically by an electronic device via any one of a plurality of geolocation techniques other than the global navigation satellite system, such as multilateration of radio signals, Global System for Mobile Communication (GSM), location based services of a mobile device, Wi-Fi based location, hybrid positioning system, and the like.

In some embodiments, the assessment may be provided via a notification or alert over the network as described elsewhere herein. For example, such a notification or alert can be provided to an electronic device described herein, including via text message, via email, via social media and/or via an application usable on the electronic device.

In some embodiments, providing the user with the assessment may comprise providing the user with one or more suggested preventative measures that reduce a rate of progression of the at least one disease in the destination and/or waypoints. Such preventative measures may be any preventative measure as described elsewhere herein. For example, Such preventative measures may be seeking immunization against the disease, taking preemptive medications that inhibit contracting and/or progression of a disease, avoiding travelling to the particular geographic location (including the destination and/or the waypoints); change the mode of transportation (such as avoiding one or more modes of transportation that cause higher risk of contracting a disease); wearing personal protective equipment in the particular geographic location, (including the destination and/or the waypoints); enhanced personal hygiene measures. In some embodiments, providing the user with the assessment may comprise suggesting that the user avoid travelling to the destination. In some embodiments, providing the user with the assessment may comprise suggesting that the user avoid travelling via at least one waypoint of the one or more waypoints. In some embodiments, providing the user with the assessment may comprise suggesting that the user travel to a different destination.

In some embodiments, the database may further comprise an indication of the at least one disease. As is discussed above, the disease database may include an indication of a least one disease. Non-limiting examples of such an indication include identifying information for a disease (e.g., disease name), identifying information for at least one pathogen (e.g., a bacterial pathogen (including bacteria described elsewhere herein), a viral pathogen (including viruses described elsewhere herein)) associated with a disease, identifying information for at least one symptom associated with the disease and a biochemical profile (e.g., biochemical profile of a bodily fluid, biochemical profile of a tissue sample) associated with the disease. In some embodiments, the indication of the at least one disease comprises identifying information for at least one virus, at least one bacterium and/or at least one protozoan.

In some embodiments, the at least one virus may be selected from the group consisting of human immunodeficiency virus I (HIV I), human immunodeficiency virus II (HIV II), orthomyxovirus, Ebola virus, Dengue virus, influenza virus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox, adenovirus, Varicella Zoster virus, Human papilloma virus (HPV), Human T-cell Leukemia Virus (HTLV), mumps virus, Respiratory Syncytial Virus (RSV), parainfluenza virus, Rubella virus, Zika virus, Middle East respiratory syndrome (MERS) Virus, Yellow Fever virus, Rift Valley fever virus, Chikungunya Fever virus, enterovirus, Cosksackie virus, and norovirus.

In some embodiments, the at least one bacterium may be selected from the group consisting of Bordetella pertussis, Chlamydia pneumoniae, Chlamydia trachomatis, Campylobacter jejuni, Helicobacter pylori, Borrelia bacteria, Mycoplasma pneumoniae, Mycobacterium tuberculosis Haemophilus influenzae, Streptococcus pyogenes, Streptococcus pneumoniae, Clostridium tetani, Treponema pallidum, Trypanosoma cruzi, Toxoplasma gondii, Yersinia pestis, and Salmonella sp.

In some embodiments, the at least one protozoan may be selected from the group consisting of Plasmodium and Leishmania donovani.

In some embodiments, the identity may include at least one of a name, age and sex of the user. Moreover, the identity may include any other suitable identification information that allows the user to be identified. Non-limiting identification information may include biometric information such as fingerprint, palm veins, face recognition, DNA, palm print, hand geometry, iris recognition, retina and odor/scent.

In some embodiments, the physiological state may include at least one of a heart rate, blood pressure, coughing frequency, coughing intensity, sneezing frequency, sneezing intensity, a level of chest congestion, a level of nasal congestion, body temperature, sweat level, weight, height, breathing rate, blood pressure, nerve conduction velocity, lung capacity, urine production rate, defecation rate, the presence of enlarged lymph nodes and a biochemical profile of a bodily fluid of the user.

In some embodiments, the method may further comprise providing the total risk of contracting the at least one disease of travelling via the waypoints to the destination. The total risk may be obtained by using statistical analysis on the risk of contracting the at least one disease at various waypoints as well as at the destination. For example, the events of contracting the at least one disease at various waypoints as well as at the destination may be considered as independent among one another. Accordingly, the total risk may be calculated as the combined probability of contacting the at least one disease at at least one geographic location among the various waypoints and the destination. Of course, if the underlying model of combined probability is different (for example, the events of contracting the at least one disease at various waypoints as well as at the destination are interdependent to some extent), the algorithm may be altered to account for it. In some embodiments, the risk of contracting the at least one disease during the journey between the starting point, the waypoints and the destination may also be taken into account in the calculation of the total risk.

The risk of contracting the at least one disease during the journey between geographic locations may be assessed qualitatively or quantitatively. In cases where the assessment is provided with a quantitative measure, one or more computational algorithms may be used to compute the quantitative measure. In some cases, the disease progression information retrieved during the search of the disease database can be used in computations. In some cases, the mode of transportation by which the journey is made may be taken into consideration in the assessment, as described elsewhere herein.

In some embodiments, the search query may further include information regarding the itinerary of travelling via the waypoints to the destination. The itinerary may include the time of arrival at each waypoint or the destination, the time of departure from each waypoint or the starting point, and/or the time of stay at each waypoint. In some cases, the itinerary may further include the mode of transportation used along the travel, such as that used from the starting point to the first waypoint, from one waypoint to the next waypoint, from the last waypoint to the destination, or the like. If there is no waypoint, the itinerary may include the time of departure from the starting point and the time of arrival at the destination. In some cases, the itinerary may further include the mode of transportation used from the starting point to the destination, between waypoints, from the starting point to a waypoint, and/or from a waypoint to the destination.

The mode of transportation may be any suitable mode for transporting a passenger from one geographic location to another. Non-limiting examples of mode of transportation include driving, coach, train, airplane, ferry, and the like.

In some embodiments, providing the user with the assessment of the risk of contracting the at least one disease may further comprise taking into account the itinerary. The itinerary may be processed, for example, by a computer processor, to allow the future geographic locations of the passenger to be determined. This may be advantageous because it may be determined based on the disease progression information that a disease may progress or regress at the future geographic locations when the passenger is schedule to stay, arrive at, or depart from the geographic locations. By taking this type of information into account, the risk of contracting the at least one disease at the future geographic locations may be determined in a more accurate or precise manner.

For example, if the itinerary shows that the passenger will arrive at waypoint A three days later, while the disease progression information indicates a disease will regress or disappear at waypoint A in two days, then it may be determined that the risk of contracting the disease at waypoint A will be low.

Moreover, the information regarding mode of transportation in the itinerary may also allow determination of the risk of contracting the at least one disease during journey between geographic locations in a more accurate or precise manner. For example, it may be determined a certain mode of transportation results in a higher risk of contracting the at least one disease during journey than another mode of transportation. For some modes of transportation that require one or more stops for embarking and discharging passengers, the disease progression information at the stops may be taken into consideration in determining the risk of contracting the at least one disease during journey.

In some aspect, the waypoints to the destination may not be entered by the user, but determined by a computer processor. That is, a route is determined from the starting point to the destination. Therefore, in another aspect among the aspects, the present disclosure provides a method for providing a user with an assessment of a risk of contracting at least one disease. The method may comprise receiving, over a network, a search query of a user, which search query includes information related to a starting point and a destination selected by the user. The search query may be processed with the aid of a computer processor and a travel cost data structure to (i) identify a route leading from the starting point to the destination within the travel cost data structure, and (ii) determine one or more waypoints along the route, wherein the one or more waypoints include at least the starting point and the destination, and wherein the travel cost data structure comprises geographic locations and travel cost between neighboring geographic locations. The method may further comprise using the one or more waypoints to search a disease database comprising disease progression information that is indicative of a progression or regression of the at least one disease in one or more geographic locations, including the destination, to identify the at least one disease and the disease progression information. Moreover, the method may comprise providing the user with the assessment of the risk of contracting the at least one disease at the destination or along the route based on the identified disease progression information.

The term “travel cost” as described herein, refers to a quantification of the desirability of travel between geographical locations. The higher the travel cost, the less desirable the travel between the geographical locations. In some embodiments, the travel cost may include one or more members (“travel cost components” hereinafter) that are selected from the group consisting of travel time, travel expense, travel comfort level, residence time, predictability, safety, punctuality, robustness, and combinations thereof.

The term “travel time” as described herein, refers to the travel time from one geographical location to another. The travel time is dependent on various factors, including but not limited to the mode of transportation, the “dead” time before taking the mode of transportation (for example, many airports require a passenger to check in a certain time before the plane takes off), weather, traffic condition, time of the year (for example, some route may take more time in certain part of a year than another part), and the like. Usually, the less the travel time, the lower the travel cost, and vice versa.

The term “residence time” as described herein, refers to the time that the passenger spends not in travelling, but staying at geographic locations. Residence time may be affected by the smoothness of the connection between legs of the travel. For example, if a passenger reaches a waypoint at a time when there is no mode of transportation for the passenger to take from a waypoint to the next waypoint or the destination, the passenger may have to stay at the current waypoint for the residence time before the mode of transportation for the next leg of the travel becomes available. Presence of residence time may result in some seemingly faster modes of transportation taking longer to travel (total travel time includes both the travel time and the residence time) than seemingly slower modes of transportation. Usually, the less the residence time, the lower the travel cost, and vice versa.

The term “travel expense” as described herein, refers to the expense undertaken by the passenger for travel, accommodation, food, as well as other applicable expenses. Usually, the less the travel expense, the lower the travel cost, and vice versa.

The term “travel comfort level” as described herein, refers to the comfort level enjoyed by the passenger during the travel, including during riding the mode of transportation and accommodation, as well as other factors that may affect the comfort level during the travel, such as scenic road, service on the mode of transportation and/or accommodation, preference of the passenger towards certain mode of transportation, and the like. Usually, the higher the travel comfort level, the lower the travel cost, and vice versa.

The term “punctuality” as described herein, refers to the probability of arriving at a geographic location at planned time. A certain modes of transportation may have higher punctuality than another mode of transportation. Other non-limiting factors that may affect punctuality include the nature of the geographic locations and the route therebetween, weather, geographic conditions, transportation infrastructure, and the like. Usually, the higher the punctuality, the lower the travel cost, and vice versa.

The term “safety” as described herein, refers to the probability of incident and accident free journey. In some cases, the severity of incidents and/or accidents, should they occur, may also be taken into consideration. A certain modes of transportation may have higher safety than another mode of transportation. Non-limiting factors that may affect safety include the nature of the geographic locations and the route therebetween, weather, geographic conditions, transportation infrastructure, and the like. Usually, the higher the safety, the lower the travel cost, and vice versa.

In some embodiments, the travel cost may include two or more travel cost components selected from the group, which two or more members are in a weighted combination. Each member from the aforesaid group of travel cost components may be allotted a coefficient for computing the weighted travel cost. The coefficient for each member may be determined by one or more computational algorithm, or predetermined. The coefficient for each member may be adjusted in accordance with the preference of the user. For example, a user may value short travel time over high travel comfort level or low travel expense. Accordingly, the coefficient for travel time may be allotted a relatively higher value than those allotted to the travel comfort level of travel expense for the user. In some embodiments, different sets of pre-determined coefficient may be provided to the user for selection. Each set of the different sets may represent different priorities or preferences, or may represent a balanced option. Non-limiting examples of such sets may include preference for cutting or avoiding one or more travel cost components, preference for a certain mode of transportation over another mode of transportation, or no preference.

The travel cost data structure may comprise geographic locations and travel cost between neighboring geographic locations. The travel cost data may be organized in various ways to provide the travel cost data structure. Non-limiting data structures that may be suitable for the present disclosure include abstract data structures (such as list, stack, queue, set, and the like), arrays, linked data structures, trees, graphs, and the like. The travel cost data structure, as used herein, is organized such that travel cost from one geographic location to another geographic location can be retrieved or computed.

The travel cost data structure may be a graph, such as a weighted graph. In some embodiments, the travel cost data structure may be a weighted map comprising the geographic locations as vertices and the travel cost between neighboring geographic locations as weighted edges. In some embodiments, there may be more than one weighted edges between two neighboring geographic locations, representing more than one mode of transportation. In some embodiments, the weighted edges may be directional, that is, the travel cost from one geographic location to another geographic location may be different from the travel cost of the return journey. In some cases, the weighted graph may be presented as an electronic map.

The travel cost data structure may be an array, such as a two-dimensional or three-dimensional table. In some embodiments, the travel cost data structure may be a table comprising geographic locations in columns and rows and the travel cost between neighboring geographic locations in cells. In some embodiment, the table may comprise a third dimension such as pages, wherein each page represents a mode of transportation.

The term “neighboring geographic locations” shall not be construed as limited to physically adjacent or connected geographic locations, but rather shall be understood in the context of modes of transportation. If one geographic location is connected directly by another geographic location via a certain mode of transportation without transit, the two geographic locations may be considered as “neighboring”. For example, geographic location A may be considered as neighboring geographic location B if there is at least one direct flight or non-stop train service between them, even though the geographic locations A and B may be thousands of miles apart, or located in different continents (such as in an intercontinental flight).

In some embodiments, the route leading from the starting point to the destination within the travel cost data structure may be generated by employing a pathfinding algorithm over the travel cost data structure. The pathfinding algorithm is capable of finding the shortest route from the starting point to the destination. The shortest path may be defined as having the lowest total value of the travel cost along the entire route. Alternatively, the shortest path may be defined as having the lowest total value of one or more travel cost components along the entire route as described elsewhere herein. Non-limiting examples of pathfinding algorithms may include A*, Dijkstra, BFS, DFS, Greedy, and combinations thereof.

In some embodiments, the method may further comprise creating an itinerary based on the route. The itinerary may be any itinerary as described elsewhere herein. In cases where an itinerary is provided, providing the user with the assessment of the risk of contracting the at least one disease may further comprise taking into account the itinerary as described elsewhere herein.

In some aspects, the route from the starting point to the destination may be generated by taking the risk of contracting the at least one disease along the route into consideration. For example, the risk of contracting the at least one disease in one or more geographic locations, and/or the risk of contracting the at least one disease in the journey from one geographic location to another may be taken into account when determining the route.

In some cases, the risk of contracting the at least one disease in two geographic locations linked by a journey, and/or the risk of contracting the at least one disease in the journey from one geographic location to another may be treated as a travel cost component from the first geographic location to the second (“disease risk” hereinafter). The disease risk may be combined with one or more other travel cost components in a weighted combination to compute an adjusted travel cost. For example, the disease risk may be allotted a coefficient and incorporated into the travel cost. In cases where more than one disease is considered, disease risk for each disease is treated as individual travel cost components for incorporation into the travel cost.

In some aspects, the travel cost data structure may be optimized by taking disease risk into consideration. Therefore, in another aspect among some aspects, the present disclosure provides a method for optimizing a travel cost data structure comprising a plurality of geographic locations and travel cost data structure between neighboring geographic locations. The method may comprise using each geographic location of the plurality of geographic locations to search a disease database comprising disease progression information that is indicative of a progression or regression of at least one disease in one or more geographic locations, to identify at least one disease and disease progression information associated with the geographic location of the at least plurality of geographic locations. The method may further comprise based on the at least one disease and disease progression information identified, (i) determining a risk of contracting the at least one disease, and (ii) optimizing the travel cost data structure by adjusting the travel cost between the each geographic location of the plurality of geographic locations and all geographic locations based on the risk. The method may further comprise repeating the aforesaid steps until all geographic locations of the plurality of geographic locations have been traversed, thereby optimizing the travel cost data structure. The optimized travel data structure may be used to generate a route from a starting point to a destination, in some cases, by using a pathfinding algorithm as described elsewhere herein.

In another aspect among some aspects, the present disclosure involves a method for providing a user with an itinerary to a destination using an optimized travel cost data structure. The method may comprise receiving, over a network, a search query of a user, which search query includes information related to a starting point and a destination selected by the user. The method may further comprise processing, with the aid of a computer processor and the optimized travel cost data structure, the search query to identify an optimum route leading from the starting point to the destination within the travel cost data structure. The method may further comprise using the optimum route to generate an itinerary for the user.

The optimum route and/or the itinerary to assess the risk of contracting the at least one disease along the route and/or at the destination. Therefore, in some embodiments, the method further comprises using each waypoint of the one or more waypoints to search a disease database comprising disease progression information that is indicative of a progression or regression of the at least one disease in one or more geographic locations, including the destination, to identify the at least one disease and the disease progression information. The method may further comprise providing the user with the assessment of the risk of contracting the at least one disease at the destination or along the route based on the disease progression information identified.

In some embodiments, providing the user with the assessment of the risk of contracting the at least one disease may further comprise taking into account the itinerary as described elsewhere herein.

The disease risk differs from many other travel cost components in several important aspect. The disease risk is transient and its level may change quickly in a period of weeks, even days or shorter. Moreover, the disease risk is less predictable than many other travel cost components. For example, travel time may vary due to completion of transportation infrastructure or change of weather in the future, but it can usually be projected in months, or even years in advance. On the contrary, it may be difficult to estimate the disease progression information in any particular geographic location in even the near future. Further, a travel cost data structure may comprise millions of data regarding geographic locations and travel cost between neighboring geographic locations. Keeping such a travel cost data structure updated with the latest disease progression information may not be cost efficient, depending on the frequency of search queries made by a user.

Therefore, in some aspect, the present disclosure provides alternative methods for generating the route from the starting point to the destination, and/or the itinerary.

In some aspects, the travel cost data structure is not optimized before generating the route. Rather, a route is first generated without taking into account the disease risk. After the route is generated, it is determined whether the route traverses any waypoint(s) where the disease risk has to be taken into consideration. If so, the travel cost data structure is only optimized at such waypoint(s). A new route may then be generated using the optimized travel cost data structure. This process may be an iterative process if the new route thus generated traverses any new waypoint(s) where the disease risk has to be taken into consideration. That is, the process is repeated again and again as necessary, for example, until no more optimization of any waypoint is needed or the process has repeated up to a threshold of times.

Therefore, in another aspect among some aspects, the present disclosure provides a method for providing a user with an itinerary to a destination. The method may comprise receiving, over a network, a search query of a user, which search query includes information related to a starting point and a destination selected by the user. The search query may be processed with the aid of a computer processor and a travel cost data structure to (i) identify a route leading from the starting point to the destination within the travel cost data structure, and (ii) determine a plurality of waypoints along the route, wherein the plurality of waypoints includes at least the starting point and the destination, and wherein the travel cost data structure comprises geographic locations and travel cost between neighboring geographic locations. The method may further comprise using each waypoint of the plurality of waypoints to search a disease database comprising disease progression information that is indicative of a progression or regression of the at least one disease in one or more geographic locations to identify the at least one disease and the disease progression information associated with the waypoint of the plurality of waypoints. The method may further comprise based on the disease progression information identified in (c), (i) determining a risk of contracting the at least one disease, and (ii) optimizing the travel cost data structure by adjusting the travel cost between the geographic location associated with the waypoint and neighboring geographic locations based on the risk. The method may further comprise repeating the aforesaid steps as necessary, to generate an optimum route, wherein the optimum route that reduces the risk of contracting the at least one disease. The method may further comprise using the optimum route to generate an itinerary for the user.

In some embodiments, the user may be provided with the itinerary on a graphical user interface as described elsewhere herein. For example, the GUI may be a component of an electronic display of an electronic device as described elsewhere herein. In some embodiment, the electronic device may be a portable electronic device. In some embodiment, the graphical user interface may be provided by a mobile computer application.

In some embodiments, providing the user with the itinerary may further comprise providing the user with an assessment of a risk of contracting at least one disease as described elsewhere herein. For example, the assessment may be provided via a notification or alert over the network as described elsewhere herein. For example, such a notification or alert can be provided to an electronic device described herein, including via text message, via email, via social media and/or via an application usable on the electronic device. In some embodiments, providing the user with the assessment may comprise providing the user with one or more suggested preventative measures that reduce a rate of progression of the at least one disease in the destination and/or waypoints as described elsewhere herein.

In some embodiments, providing the user with the assessment may comprise suggesting that the user avoid travelling to the destination. A threshold of the number of reiteration of the method may be predetermined. Moreover, threshold of the travel cost may be predetermined. If the reiteration of the method reaches the threshold number, without the level of the total travel cost dropping below the threshold of the travel cost, the method may be terminated and the user may be suggested to avoid travelling to the destination. Alternatively, a threshold of disease risk may be pre-determined. If the reiteration of the method reaches the threshold number, without the level of the disease risk along the route dropping below the threshold of the travel cost, the method may be terminated and the user may be suggested to avoid travelling to the destination. In some embodiments, providing the user with the assessment may comprise suggesting that the user travel to a different destination.

In some aspect, not just one route, but a plurality of routes will be chosen first to determine whether the routes traverse any waypoint(s) where the disease risk has to be taken into consideration. By employing a plurality of routes, waypoints that are affected by at least one disease can be identified more quickly, which may allow the optimum route to be identified more quickly. Therefore, a plurality of routes is first generated without taking into account the disease risk. After the routes are generated, it is determined whether the routes traverse any waypoint(s) where the disease risk has to be taken into consideration. If so, the travel cost data structure is only optimized at such waypoint(s). One or more new routes may then be generated using the optimized travel cost data structure. This process may be an iterative process if the new route(s) thus generated traverse(s) any new waypoint(s) where the disease risk has to be taken into consideration. That is, the process is repeated again and again as necessary, for example, until no more optimization of any waypoint is needed or the process has repeated up to a threshold of times.

The number of routes generated in each reiteration of the method may be the same. Alternatively, the number of routes generated in each reiteration of the method may be different. For example, in each reiteration of the method, 2, 3, 4, 5, 6, 7, 8, 9, 10, 11, 12, 13, 14, 15, 20, 25, 30, 35, 40, 45, 50, 100, 200, 300, 500, 1000, or more than 1000 routes, or any integer number of routes between the numeric value as enumerated above may be generated.

In some embodiments, the plurality of routes may be randomly chosen. Alternatively, the plurality of routes may be those ranked with the lowest travel cost among available routes.

Therefore, in another aspect among some aspects, the present disclosure involves a method for providing a user with an itinerary to a destination. The method may comprise receiving, over a network, a search query of a user, which search query includes information related to a starting point and a destination selected by the user. The search query may be processed with the aid of a computer processor and a travel cost data structure to (i) identify a plurality of routes leading from the starting point to the destination within the travel cost data structure, and (ii) for each route of the plurality of routes, determine a plurality of waypoints along the route, wherein the plurality of waypoints includes at least the starting point and the destination, and wherein the travel cost data structure comprises geographic locations and travel cost between neighboring geographic locations. For each route of the plurality of routes, each waypoint of the plurality of waypoints may be used to search a disease database comprising disease progression information that is indicative of a progression or regression of the at least one disease in one or more geographic locations to identify the at least one disease and the disease progression information associated with the waypoint of the plurality of waypoints. The method may further comprise, based on the disease progression information identified, for each route of the plurality of routes, (i) determining a risk of contracting the at least one disease along the route, and (ii) optimizing the travel cost data structure by adjusting the travel cost between the geographic location associated with the waypoint and neighboring geographic locations based on the risk. The method may further comprising repeating the aforesaid steps as necessary, to generate an optimum route, wherein the optimum route incurs the lowest travel cost among the plurality of routes. The method may further comprise using the optimum route to generate an itinerary for the user.

The method may further comprise providing a plurality of routes (e.g., optimum routes) and/or itineraries for the user to select from. The plurality of routes and/or itineraries may be those having the lowest total travel cost. Alternatively, the plurality of routes and/or itineraries may each be one with the lowest total travel cost according to individual preference settings. Each preference setting may correspond to a different set of coefficients. For example, the user may be presented with itineraries labelled as “preference for short travel time”, “preference for cheap travel expense”, “preference for low disease risk”, “no preference”, and the like, from which the user may choose from.

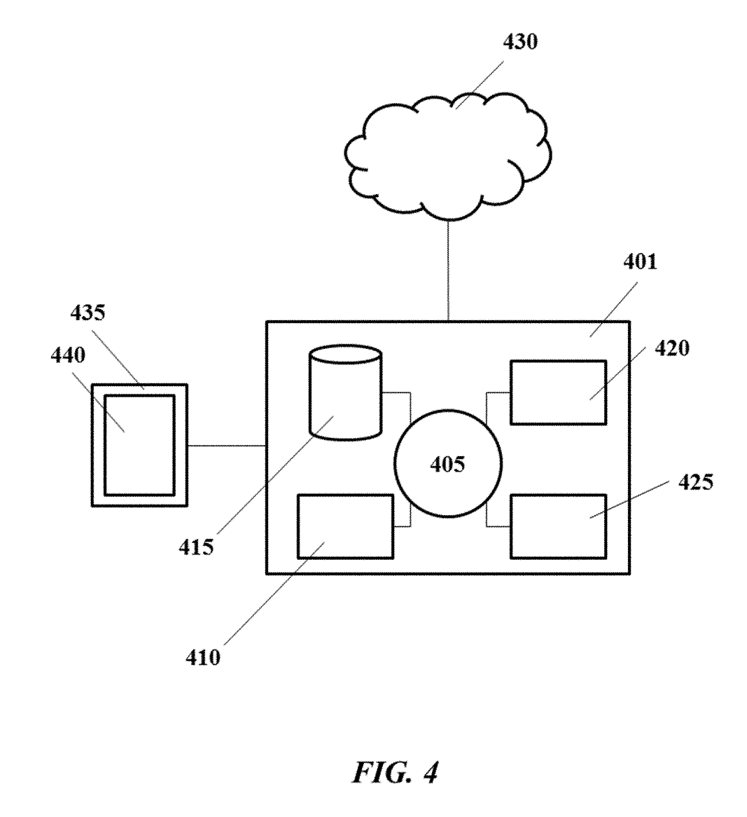

The present disclosure provides computer control systems that are programmed to implement methods of the disclosure. FIG. 4 shows an example computer system 401 that can be programmed or otherwise configured in a number of ways, including to process a search query of a user; contain a disease database; generate a quantitative measure of a biological marker from nucleic acid amplification data; process a quantitative measure of a biological marker to obtain disease information indicative of progression or regression of a disease; process such disease information to obtain a trend and/or correlation; assess risk of contracting a disease; and/or displaying information to a user. The computer system 401 can regulate various aspects of biological sample processing via nucleic acid amplification, such as, for example, amplification protocols that are executed by a thermocycler or other type of amplification device. The computer system 401 can be an electronic device of a user or a computer system that is remotely located with respect to the electronic device. The electronic device can be a mobile electronic device.

The computer system 401 includes a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet. The network 430 in some cases is a telecommunication and/or data network. The network 430 can include one or more computer servers, which can enable distributed computing, such as cloud computing. The network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC).

The storage unit 415 can store files, such as drivers, libraries and saved programs. The storage unit 415 can store user data, e.g., user preferences and user programs. The computer system 401 in some cases can include one or more additional data storage units that are external to the computer system 401, such as located on a remote server that is in communication with the computer system 401 through an intranet or the Internet.

The computer system 401 can communicate with one or more remote computer systems through the network 430. For instance, the computer system 401 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods as described herein can be implemented by way of machine (e.g., computer processor) executable code stored on an electronic storage location of the computer system 401, such as, for example, on the memory 410 or electronic storage unit 415. The machine executable or machine readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display 435 that comprises a user interface (UI) 440 for providing, for example, information (e.g., disease information, disease trends, recommendations for treatment of a disease, recommendations for prevention of a disease, a questionnaire, a report as described elsewhere herein, an alert/notification, or any other type of information described elsewhere herein). The electronic display 435 may be part of a mobile electronic device (e.g., portable computer, smart phone, or tablet personal computer) of the user. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 405. The algorithm can, for example, determine quantitative measures of biological markers from nucleic acid amplification data; process quantitative measures to obtain disease information indicative of the progression or regression of a disease; process disease information to generate a disease trend; determining an update to a trend; providing an assessment of a risk of contracting a disease; determining a correlation(s) between results of a questionnaire and a disease; and processing a search query and searching a disease database.

### EXAMPLES

### Example 1

### Disease Risk Assessment

A user located in San Francisco, Calif. accesses a mobile application on his or her smartphone. The mobile application provides the user with a graphical user interface having a search field in which the user can enter a string of keywords that is used as a search query. The user enters the keywords “chest congestion” “body temperature 39° C.” and “San Francisco, Calif.” and clicks a “search” button near the search field. The smartphone transmits the keywords to a remote computer system, over the wireless network to which the smartphone is connected/the Internet, whereby the remote computer system receives the keywords. With the aid of its computer processor, the remote computer system processes the keywords and identifies the tags “congestion”, “39° C.” and “San Francisco” as tags that are usable to search a disease database that is stored in memory of the remote computer system.

Using the tags identified above, the computer processor searches the disease database using the tags and identifies “congestion”, “39° C.” and “San Francisco” as associated with the Influenza B virus. The computer processor also identifies information indicative of a relatively high rate of prevalence of Influenza B virus among 25-35years olds in San Francisco. The prevalence information is supplied to the database by disease monitoring data obtained from age 25-35 users in San Francisco. Based on the relatively high prevalence, the computer processor calculates a risk assessment that includes a quantitative score indicative of a relatively high risk of the user contracting Influenza B/a relatively high likelihood that the user has Influenza B virus. The risk assessment is transmitted back to the smart phone over the network, where the mobile application displays it to the user. Along with the risk assessment, the mobile application displays to the user preventive measures that can be taken to avoid contracting Influenza B (e.g., washing hands regularly, use of hand sanitizer, wearing a mask over the user's nose and mouth, getting a vaccination against Influenza B, etc.) and/or to treat Influenza B and its symptoms (e.g., taking anti-inflammatory drugs to reduce fever/pain, drinking plenty of liquid, taking one or more immunostimulants (e.g., Vitamin C), getting sufficient rest, etc.).

### Example 2

### Disease Monitoring in a Subject

A subject separately provides each of a plurality of 0.1 mL whole blood samples obtained at differing time points directly to the reaction vessel of a point-of-care (POC) device. The whole blood samples are not subjected to purification to isolate nucleic acids from the whole blood samples. The POC device also includes a heater that cycles the temperature of a reaction mixture in the reaction vessel, an optical detector for detecting reaction products generated in the reaction vessel and on-board electronics that process detection data into an amount of a biological marker in the reaction mixture, based upon detected amplification products. The POC device also includes an electronic display that includes a GUI that both permits the subject or another user to control nucleic acid amplification and displays various forms of information (e.g., disease information, etc.) and other items (e.g., questionnaires) to the subject or other user such as a healthcare professional as described elsewhere herein.

H3N2 Influenza virus is identified as a disease of interest via the subject's answers to a questionnaire provided by the POC to the subject, such answers including the subject's age, sex, geographic location and symptoms. Accordingly, the reaction vessel contains a reaction mixture that comprises, in addition to a given whole blood sample, reagents necessary for amplification of any nucleic acid biological markers indicative of H3N2 Influenza virus. The reagents include a reverse transcriptase, DNA polymerase, nucleotides and one or more primer(s) with sequence homology to sequences specific to H3N2 Influenza virus RNA. The reaction mixture also includes a TaqMan probe targeted to amplification products that can be used for optical detection of amplification products as described elsewhere herein. Each whole blood sample obtained from the subject is processed separately in the POC device.

Upon initiation of thermocycling, H3N2 Influenza nucleic acid is reverse transcribed via the action of the reverse transcriptase and the resulting DNA transcripts subsequently amplified via the action of the DNA polymerase (e.g., an RT-PCR process) to form amplified products indicative of H3N2 Influenza nucleic acid biological markers in the sample. Nucleic acid amplification is achieved in less than 10 minutes, often less than 5 minutes. During amplification, signal from the released optical dye of the TaqMan probe is detected and the amount of amplification products determined. An on-board computer processor of the POC used the amount of amplification and amplification cycle number to determine the amount of H3N2 Influenza nucleic acid in the given whole blood sample.

The on-board computer processor then processes the amounts of H3N2 nucleic acid biological marker obtained at the various time points by comparing them amongst one another and to a baseline biological marker amount stored in the POCs memory. The baseline biological marker amount corresponds to an amount of nucleic acid biological marker indicative of a healthy state, not considered to be associated with H3N2 Influenza virus. In this particular example, the amount of H3N2 in the subject's blood increases over the multiple time points tested and is statistically higher in value than the healthy amount at all time-points tested. Accordingly, the computer processor determines that H3N2 Influenza virus has progressed in the subject. An output of this disease information is provided to the subject or another user (e.g., a healthcare professional as described elsewhere herein), such as on the electronic display of the POC device. The output can also include a determined correlation between one or more of the subject's answers to the questionnaire and the disease information such, as for example, the progression of the H3N2 Influenza virus in the subject and the subject's geographic location. In some cases, the output is transmitted, over a network, to a remote computer storage system for later retrieval and use.

### Example 3

### Disease Monitoring Across Subjects

The prevalence of Streptococcus pneumoniae infection is monitored across the San Francisco Bay Area, including the cities of San Jose, Calif., San Francisco, Calif. and Oakland, Calif. Each of a plurality of subjects located in a particular geographic location in the San Francisco Bay Area separately provides each of a plurality of 0.1 mL saliva samples obtained at differing time points directly to the reaction vessel of a POC device. A plurality of POC devices are used to process samples from the various subjects. The saliva samples are not subjected to purification to isolate nucleic acids from the saliva samples. Each POC device also includes a heater that cycles the temperature of a reaction mixture in the reaction vessel, an optical detector for detecting reaction products generated in the reaction vessel and on-board electronics that process detection data into an amount of a biological marker in the reaction mixture, based upon detected amplification products. Each POC device also includes an electronic display that includes a GUI that both permits the subject or another user to control nucleic acid amplification and displays various forms of information (e.g., disease information, etc.) and other items (e.g., questionnaires) to the subject or other user such as a healthcare professional as described elsewhere herein. Moreover, each POC device is in electronic communication with a remote computer system that stores information obtained from the POC devices.

In each POC device, the reaction vessel contains a reaction mixture that comprises, in addition to a given saliva sample, reagents necessary for amplification of any nucleic acid biological markers indicative of Streptococcus pneumoniae. The reagents include a DNA polymerase, nucleotides and one or more primer(s) with sequence homology to sequences specific to Streptococcus pneumoniae DNA. The reaction mixture also includes a TaqMan probe targeted to amplification products that can be used for optical detection of amplification products as described elsewhere herein. Each saliva sample obtained from a subject is processed separately in a POC device.

Upon initiation of thermocycling, Streptococcus pneumoniae nucleic acid is amplified via the action of the DNA polymerase (e.g., a PCR process) to form amplified products indicative of Streptococcus pneumoniae nucleic acid biological markers in the given saliva sample. Nucleic acid amplification is achieved in less than 10 minutes, often less than 5 minutes. During amplification, signal from the released optical dye of the TaqMan probe is detected and the amount of amplification products determined. An on-board computer processor of the POC used the amount of amplification and amplification cycle number to determine the amount of Streptococcus pneumoniae nucleic acid in the given saliva sample.

For each subject, the on-board computer processor of a POC device then processes the amounts of Streptococcus pneumoniae nucleic acid biological marker obtained at the various time points by comparing them amongst one another and to a baseline biological marker amount stored in the POCs memory. The baseline biological marker amount corresponds to an amount of nucleic acid biological marker indicative of a healthy state, not considered to be associated with Streptococcus pneumoniae. For example, the amount of Streptococcus pneumoniae in the subject's blood may increase over the multiple time points tested and may be statistically higher in value than the healthy amount at all time-points tested. Accordingly, the computer processor determines that Streptococcus pneumoniae has progressed in the subject. In parallel or at different times, saliva samples are processed for the other subjects and Streptococcus pneumoniae progression/regression information determined for each other subject.

The Streptococcus pneumoniae progression/regression information obtained from the various subjects is transmitted over a network, such as a wireless network/the Internet, from the POC devices to a remote computer system that compiles and stores the collected information in its computer memory. A computer processor of the remote computer system then processes the disease information to identify a trend of Streptococcus pneumoniae in the San Francisco Bay Area. In this particular example, information from a majority of the subjects analyzed showed a progression of Streptococcus pneumoniae with increasing amounts of Streptococcus pneumoniae biological marker in saliva samples over time and at statistically higher levels than reference. Accordingly, the computer processor generates a trend of increasing prevalence of Streptococcus pneumoniae in the San Francisco Bay Area.

An output of the trend is provided to a user(s) (e.g., one or more of the subjects, a healthcare professional as described elsewhere herein), such as on the electronic display of a POC device or mobile computing device. The output can be provided to the user as a notification or alert, e.g., such as a text message, email or page, prompting the user to take appropriate medical action (if any). In some cases, the output of the trend is stored in a memory location for later retrieval and use. The output can be stored on the remote computer system, transmitted over the network back to one or more of the POC devices or transmitted over the network back to one or more other remote computer systems.

The analysis is then repeated with a plurality of second subjects, which can be the same plurality of subjects as the first plurality of subjects analyzed; a group that includes at least a subset of the first plurality of subjects analyzed; or entirely different group of subjects from the San Francisco Bay Area. Disease information is processed to obtain a trend that shows an even greater rate of disease progression, which includes an increase in the prevalence of Streptococcus pneumoniae in the San Francisco Bay Area. The trend is outputted to one or more user(s) as described above for further attention and action.

### Example 4

### Generation of Itinerary and Risk Assessment

A user located in Beijing, China accesses an application on his or her tablet computer. The application provides the user with a graphical user interface having a search field in which the user can enter a string of keywords that is used as a search query. The search field is labelled as “destination”. The user enters the keywords “Serengeti”. The tablet computer determines the location of the user automatically using multilateration of radio signals among multiple cell towers of the data network the tablet computer is connected. The tablet computer then transmits the keyword together with the geographic location of the user to a remote computer system, over the data network, whereby the remote computer system receives the keywords via Internet to which the data network is connected. With the aid of its computer processor, the remote computer system processes the keyword and the geographic location of the user and identifies the geographic location tags “Beijing, China” and “Serengeti National Park, Tanzania” as tags that are usable to search an electronic map that is stored in memory of the remote computer system.

Using the aforesaid geographic location tags, the computer processor identifies a route from the starting point “Beijing, China” to the destination “Serengeti National Park, Tanzania” which includes three waypoints, namely Abu Dhabi, UAE (the United Arab Emirates), Dar es Salaam, Tanzania, and Serengeti National Park, Tanzania. The computer processor then uses the three waypoints to search the disease database and identifies “Dar es Salaam, Tanzania” as associated with disease progression information regarding Zika virus endemic. Based on the disease progression information, the computer processor determines that there is a high prevalence of Zika virus endemic in Dar es Salaam, Tanzania recently, and the probability of regression of the Zika virus endemic is low in the near future. The computer processor also calculates a risk assessment that indicates a relatively high risk of the user contracting Zika virus. Based on the risk assessment, the computer processor recalculates the travel cost associated with Dar es Salaam, Tanzania, thereby optimizing the electronic map.

The computer processor then identifies a second route from the starting point “Beijing, China” to the destination “Serengeti National Park, Tanzania” using the optimized electronic map. The second route comprises four waypoints, namely Hong Kong, Dubai, UAE, Nairobi, Kenya, and Serengeti National Park, Tanzania. The computer processor then uses the four waypoints to search the disease database and identifies “Nairobi, Kenya” as associated with disease progression information regarding AIDS. Based on the disease progression information, the computer processor calculates a risk assessment that indicates a low risk of the user contracting AIDS. Based on the risk assessment, the computer processor recalculates the travel cost associated with Nairobi, Kenya, thereby optimizing the electronic map.

The computer processor then attempts to identify a third route from the starting point “Beijing, China” to the destination “Serengeti National Park, Tanzania” using the optimized electronic map. The third route is the same as the second route. The computer then determines no further optimization is needed and that the third route is the optimum route.

The computer processor then generates an itinerary based on the optimum route. The computer processor further calculates a risk assessment that includes a quantitative score indicative of a low risk of the user contracting AIDS along the route. The itinerary and the risk assessment are transmitted back to the tablet PC over the data network, where the application displays it to the user on the screen of the tablet PC.

### Example 5

### Generation of Itinerary and Risk Assessment

A group of tourists are stuck in a resort A on an island B during an endemic of a certain disease C. The tourists want to get to the only airport D in the island B to leave this island. One tourist accesses an application on his or her laptop. The application provides the user with a graphical user interface having at least two search fields in which the user can enter a string of keywords that is used as a search query. One search field is labelled as “starting point” and another search field is labelled as “destination”. The user enters the keyword “resort A” in the first search field and the keyword “airport D” in the second search field. The laptop transmits the keywords to a remote computer system, over a wired network connected to the Internet, whereby the remote computer system receives the keywords. With the aid of its computer processor, the remote computer system processes the keywords and identifies the geographic location tags associated with the resort A and the airport D that are usable to search an electronic map that is stored in memory of the remote computer system.

Using the aforesaid geographic location tags, the computer processor identifies five routes (E1 to E5) with the lowest total travel cost from the starting point “resort A” to the destination “airport D”. E1 to E3 all involves getting to a coach station F first and taking different bus routes to the airport D. E4 involves getting to a port G and taking boat to the airport D. E5 involves walking to a train station H close to the resort A and taking a train to a bus stop I close to the airport D, and takes a bus to the airport.

The computer processor then uses the aforesaid waypoints F to Ito search the disease database and identifies all of them as associated with disease progression information regarding the disease C. Based on the disease progression information, the computer processor calculates risk assessment of contracting the disease C at each waypoint, taking into the mode of transportation to and from these waypoints into consideration. Based on the risk assessment, the computer processor recalculates the travel cost associated with each waypoint, thereby optimizing the electronic map.

The computer processor then identifies five new routes (J1 to J5) with the lowest total travel cost from the starting point “resort A” to the destination “airport D” using the optimized electronic map. All five new routes (J1 to J5) involve getting to a limousine company K and renting a limousine to the airport D. The computer processor uses the waypoint K to search the disease database and identifies it as associated with disease progression information regarding the disease C. Based on the disease progression information, the computer processor calculates risk assessment of contracting the disease C at each waypoint, taking into the mode of transportation to and from these waypoints into consideration. Based on the risk assessment, the computer processor recalculates the travel cost associated with the waypoint K, thereby optimizing the electronic map.

The computer processor then repeats the reiteration of the process several times, resulting in five routes (L1 to L5) with the lowest total travel cost from the starting point “resort A” to the destination “airport D”, while further reiteration of the process does not recognize any new waypoint as associated with any disease progression information. However, the computer processor determines that none of the five routes (L1 to L5) incurs a travel cost below a predetermined threshold for travel cost.

The computer processor then determines that there is a high probability that the purpose of the user is to find a way out of the island based on search patterns from other users stored in a memory of the remote computer system. The computer processor then determines that a ferry M may serve the purpose of the user as well. The computer processor then uses the ferry M as the destination and repeats the aforesaid process several times and identifies five routes (N1 to N5) with the lowest total travel cost from the starting point “resort A” to the destination “ferry M”.

The computer processor then generates an itinerary based on each of the five routes. The computer processor further calculates a risk assessment that includes a quantitative score indicating the risk of the user contracting the disease C along each route. The itineraries and the risk assessment are transmitted back to the laptop over the wired network, together with a notification suggesting that the user avoid travelling to the airport D and a notification suggesting that the user travel to the ferry M, where the application displays it to the user on the screen of the laptop for the user to choose between the five itineraries.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-228. (canceled)

229. A method for providing a user with an assessment of a risk of contracting at least one disease, comprising:
(a) receiving, over a network, a search query of a user, which search query includes information related to at least any two of an identity, a geographic location and a physiological state of said user;
(b) processing, with the aid of a computer processor, said search query to identify one or more tags that are usable for searching in a disease database, wherein said disease database comprises (i) an indication of said at least one disease, (ii) disease progression information that is indicative of a progression or regression of said at least one disease in one or more geographic locations, (iii) subject information selected from two or more of an identity, geographic location and physiological state of each of a plurality of subjects, and (iv) one or more associations between said at least one disease, disease progression information and subject information;
(c) searching said disease database using said one or more tags to identify said at least one disease and said disease progression information; and
(d) based on said disease progression information, providing said user with said assessment of said risk of contracting said at least one disease.

230. The method of claim 229, wherein said user is provided with said assessment of said risk of contracting said at least one disease on a graphical user interface on an electronic display of an electronic device.

231. The method of claim 230, wherein: (1) said electronic device is a portable electronic device or (2) said graphical user interface is provided by a mobile computer application.

232. The method of claim 229, wherein said information is related to said identity, geographic location and physiological state of said user.

233. The method of claim 229, wherein said assessment is provided via a notification or alert over said network.

234. The method of claim 229, wherein providing said user with said assessment comprises providing said user with one or more suggested preventative measures that reduce a rate of progression of said at least one disease in said geographic location.

235. The method of claim 229, wherein said indication of said at least one disease comprises identifying information for at least one virus, at least one bacterium and/or at least one protozoan.

236. The method of claim 229, wherein said identity includes at least one of a name, age and sex of said user.

237. The method of claim 229, wherein said physiological state includes at least one of a heart rate, blood pressure, coughing frequency, coughing intensity, sneezing frequency, sneezing intensity, a level of chest congestion, a level of nasal congestion, body temperature, sweat level, weight, height, breathing rate, blood pressure, nerve conduction velocity, lung capacity, urine production rate, defecation rate, a presence of enlarged lymph nodes and a biochemical profile of a bodily fluid of said user.

238. The method of claim 229, wherein said geographic location is a continent, an island, a grouping of islands, a city/town/village, a county/township, a prefecture, a parish, a province, a state, a territory, an administrative region, a country, and/or a grouping of countries.

239. A method for providing a user with an assessment of a risk of contracting at least one disease, comprising:
(a) receiving, over a network, a search query of a user, which search query includes information related to a destination, and optionally one or more waypoints;
(b) processing, with the aid of a computer processor, said search query to identify one or more geographic location tags associated with the destination and optionally the one or more waypoints for searching in a disease database, wherein said disease database comprises disease progression information that is indicative of a progression or regression of said at least one disease in one or more geographic locations, including said destination;
(c) searching said disease database using said one or more geographic location tags to identify said at least one disease and said disease progression information; and
(d) based on said disease progression information identified in (c), providing said user with said assessment of said risk of contracting said at least one disease at said destination and optionally said one or more waypoints.

240. The method of claim 239, wherein: said user is provided with said assessment of said risk of contracting said at least one disease on a graphical user interface on an electronic display of an electronic device.

241. The method of claim 240, wherein: (1) said electronic device is a portable electronic device or (2) said graphical user interface is provided by a mobile computer application.

242. The method of claim 239, wherein said search query further includes an identity of said user, a starting point of said user, or a physiological state of said user.

243. The method of claim 239, wherein said assessment is provided via a notification or alert over said network.

244. The method of claim 239, wherein providing said user with said assessment comprises providing said user with one or more suggested preventative measures that reduce a rate of progression of said at least one disease in said destination and/or waypoints.

245. The method of claim 239, wherein providing said user with said assessment comprises suggesting that said user avoid travelling to said destination or travel to a different destination.

246. The method of claim 239, wherein providing said user with said assessment comprises suggesting that said user avoid travelling via at least one waypoint of said one or more waypoints.

247. The method of claim 239, wherein said database further comprise an indication of said at least one disease.

248. The method of claim 247, wherein said indication of said at least one disease comprises identifying information for at least one virus, at least one bacterium and/or at least one protozoan.

249. The method of claim 242, wherein said identity includes at least one of a name, age and sex of said user.

250. The method of claim 242, wherein said physiological state includes at least one of a heart rate, blood pressure, coughing frequency, coughing intensity, sneezing frequency, sneezing intensity, a level of chest congestion, a level of nasal congestion, body temperature, sweat level, weight, height, breathing rate, blood pressure, nerve conduction velocity, lung capacity, urine production rate, defecation rate, a presence of enlarged lymph nodes and a biochemical profile of a bodily fluid of said user.

251. The method of claim 239, further comprising providing a total risk of contracting said at least one disease of travelling via said waypoints to said destination.

252. The method of claim 239, wherein the search query further includes information regarding an itinerary of travelling via said waypoints to said destination.

253. The method of claim 252, wherein said itinerary includes a time of arrival at each waypoint or the destination, a time of departure from each waypoint or the starting point, or a time of stay at each waypoint.

254. The method of claim 253, wherein providing said user with said assessment of said risk of contracting said at least one disease in (d) further comprises taking into account said itinerary.
